(19) United States
(12) Patent Application Publication
(10) Pub. No.: US 2021/0082563 A1
(43) Pub. Date: Mar. 18, 2021

(54) METHODS FOR IMPROVING PSYCHOLOGICAL THERAPY OUTCOME

(71) Applicant: IESO DIGITAL HEALTH LIMITED, Cambridge (GB)

(72) Inventors: Ana Maria Ferreira Paradela Catarino WINGFIELD, Cambridge (GB); Mihai Valentin TABLAN, Cambridge (GB); Sarah Elisabeth BATEUP, Cambridge (GB); Andrew BLACKWELL, Cambridge (GB)

(21) Appl. No.: 16/961,399

(22) PCT Filed: Jan. 14, 2019
(86) PCT No.: PCT/GB2019/050095

§ 371 Date: Jul. 10, 2020

(30) Foreign Application Priority Data
Jan. 12, 2018 (GB) 1800556.1
Sep. 21, 2018 (GB) 1815409.6

(51) Int. Cl. G16H 20/70; G16H 10/60; G16H 10/20; G16H 50/30; G16H 50/70

(52) U.S. Cl. CPC G16H 20/70; G16H 10/60; G16H 50/70; G16H 50/30; G16H 10/20

(57) ABSTRACT

A method of determining the effectiveness of a therapist, comprising: obtaining data relating to one or more patient variables and/or one or more service variables for one or more patient suffering from a mental health disorder and allocated to the therapist; attributing a score to the data for each of the patient variables and/or the service variables, wherein the scores are based on a correlation between historic cohort treatment outcomes and historic cohort data comprising cohort patient and/or service variables; combining the scores to calculate an aggregate score; comparing the aggregate score with a scale to produce a prediction of psychological therapy outcome for the one or more patient; obtaining an observation of psychological therapy outcome for the one or more patient after treatment by the therapist has been provided; and comparing the observation of psychological therapy outcome and the prediction of psychological therapy outcome for the one or more patient to make a determination of the effectiveness of the therapist.

### BACKGROUND

The present disclosure relates to methods, particularly computer-implemented methods, for predicting psychological therapy outcomes. The disclosure also relates to methods of treating a patient suffering from a mental health disorder, and methods of determining the effectiveness of a therapist.

Common mental health disorders including depression and anxiety are characterized by intense emotional distress, which affects social and occupational functioning. About one in four adults worldwide suffer from a mental health problem in any given year. In the US, mental disorders are associated with estimated direct health system costs of $201 billion per year, growing at a rate of 6% per year, faster than the gross domestic product growth rate of 4% per year. Combined with annual loss of earnings of $193 billion, the estimated total mental health cost is at almost $400 billion per year. In the UK mental health disorders are associated with service costs of £22.5 billion per year and annual loss of earnings of £26.1 billion. For these reasons, new approaches are required to improve access to evidence-based treatment whilst managing costs.

In the past, payor costs associated with delivering care have been managed by imposed limits on service utilization, elevated insurance premiums and high co-payment rates, with allocation of patients to treatment based on availability rather than suitability. Recognizing the limitations of these approaches, alternatives are being investigated, including widespread deployment of recovery-focused clinical models, stepped/stratified care systems, outcomes-based reimbursement, and the development of personalized treatment programmes as part of a quality improvement cycle aiming to drive up standards in mental healthcare.

### BRIEF DESCRIPTION OF THE DRAWINGS

The following figures are included to illustrate certain aspects of the embodiments, and should not be viewed as exclusive embodiments. The subject matter disclosed is capable of considerable modifications, alterations, combinations, and equivalents in form and function, as will occur to those skilled in the art and having the benefit of this disclosure.

FIG. 1 illustrates a flow diagram of a computer-implemented method of the present disclosure.

FIG. 2 illustrates a timeline of the treatment protocol.

FIG. 3 is a chart of the likelihood of patient improvement based on patient health questionnaire (PHQ-9) scores and general anxiety disorder (GAD-7) scores at assessment, calculated from patients completing a course of internet-enabled CBT treatment. For a particular combination of GAD7 score at initial assessment (Y-axis) and PHQ9 score at initial assessment (X-axis), the likelihood of clinical improvement is represented as a percentage, with higher likelihoods of improvement (higher percentage) being represented by darker grey cells. Blank cells represent scores for which improvement rate is not calculable.

FIG. 4 is a chart of the likelihood of patient recovery based on PHQ-9 and GAD-7 scores at assessment, calculated from patients completing a course of internet-enabled CBT treatment. For a particular combination of GAD7 score at initial assessment (Y-axis) and PHQ9 score at initial assessment (X-axis), the likelihood of clinical recovery is represented as a percentage, with higher likelihoods of recovery (higher percentage) being represented by darker grey cells. Blank cells represent scores for which recovery rate is not calculable.

FIG. 5 is a study profile and patient flowchart relating to the data presented in Example 2.

FIG. 6 is a histogram showing the efficacy of therapists relating to the data presented in Example 3. Efficacy is determined by observed overall recovery rate (ORR) of a therapist's patients minus the expected ORR for the same patients. A negative value for observed ORR minus expected ORR indicates that the therapist is less effective than average, a positive value for observed ORR minus expected ORR indicates that the therapist is more effective than average.

FIG. 7 illustrates a flow diagram of a method of the present disclosure.

FIG. 8 illustrates a system for performing the computer-implemented methods of the present invention.

FIG. 9a illustrates a device which may form part of the system of FIG. 8.

FIG. 9b illustrates a server which may form part of the system of FIG. 8.

### DETAILED DESCRIPTION

The present disclosure relates to computer-implemented methods for predicting psychological therapy outcomes.

Internet-enabled cognitive behavioral therapy (IECBT) is a type of high-intensity online therapy used within an Improving Access to Psychological Therapies (IAPT) program. Within IAPT using IECBT, patients are offered weekly one-to-one sessions with an accredited therapist, similar to face-to-face programs whilst retaining the advantages of online services including convenience, accessibility, increased disclosure and shorter waiting times. IECBT also offers unique data monitoring opportunities to identify associations between patient and service variables to predict clinical outcomes, assign treatment protocols, and integrate with a fee-for-value payment system.

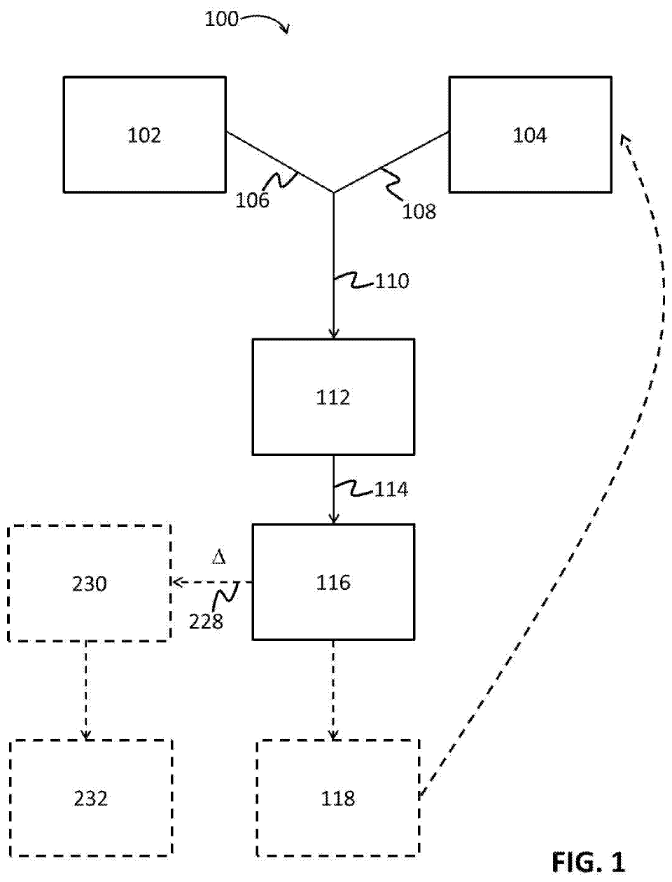

FIG. 1 illustrates a flow diagram of a computer-implemented method 100 of the present disclosure. First, one or more patient variables 102 and/or one or more service variables 104 are collected as inputs.

Exemplary patient variables 102 include, but are not limited to, (1) patient gender, (2) patient age, (3) whether or not the patient suffers from a long-term physical condition, (4) whether or not the patient is taking psychotropic medication (e.g., anti-depressants or anxiolytics) at the start of treatment, (5) the initial symptom severity, (6) the mental health disorder the patient suffers from, (7) whether or not a patient is currently pregnant, or has been pregnant or given birth in the previous 12 months and (8) patient employment status. The initial symptom severity can be ascertained using, for example but not limited to, the general anxiety disorder 7-item (GAD-7) scale, the patient health questionnaire (PHQ-9), and/or other disorder specific anxiety measures such as the Obsessive Compulsive Inventory (OCI), Impact of events scale-revised (IES-R), Agoraphobia Mobility Inventory (AMI), Social Phobia Inventory (SPI), Panic disorder severity scale (PDSS) and health anxiety inventory (HAI).

Exemplary service variables 104 include, but are not limited to, (1) waiting times between various stages in the patient journey, (2) treatment duration, (3) the number of scheduled appointments the patient fails to attend, (4) the therapist the patient is allocated to, and (5) the therapeutic protocol the patient receives.

The patient variables 102 and service variables 104 may be provided by the patient, the service provider (e.g., a doctor, a nurse, a technician, or a receptionist), or a computer or computer program. For example, a patient's attendance to scheduled appointments may be tracked using a computer or computer program where they check in and out of scheduled appointments. Then, the computer or computer program can provide the number of scheduled appointments the patient fails to attend. Alternatively, a receptionist or therapist may similarly provide the number of scheduled appointments the patient fails to attend.

Referring again to FIG. 1, after the inputs are collected, the method 100 then involves assigning a patient variables score 106 to the patient variables 102 provided and assigning a service variables score 108 to the service variables 104 provided.

Patient variables 102 and service variables 104 are assigned scores using logistic regression models, with psychological therapy outcome 116 as the variable to be predicted.

The scores associated with the patient variables 102 and the service variables 104 are summed 110 to provide an aggregate score 112, which is used 114 to yield a prediction of psychological therapy outcome 116. The use 114 of the aggregate score 112 may optionally be by e.g. comparison to an outcome scale or reference scale. The prediction of psychological therapy (e.g., IECBT treatment) outcome 116 may be a measure of improvement and/or a measure of recovery. The aggregate score 112 may be expressed as a number between 0 and 1, such that if the aggregate score 112 was close to 0, the prediction of psychological therapy outcome 116 could be that the probability of improvement and/or recovery is low, in other words a prediction of poor psychological therapy outcome; by extension, if the aggregate score 112 was close to 1, the prediction of psychological therapy outcome 116 could be that the probability of improvement and/or recovery is high, in other words a prediction of good psychological therapy outcome. An optional outcome scale or reference scale for comparison with the aggregate score 112 could for example define one or more thresholds, e.g. a threshold aggregate score below which the patient may not be predicted to recover/improve following therapy, or a threshold aggregate score above which the patient would be predicted to recover/improve following therapy. As used herein, “improvement” is defined as when a patient shows a significant reduction in at least one of the outcome measures (i.e., decrease of 4 points or more in the GAD-7 and/or 6 points or more in the PHQ-9).

As used herein, “recovery” is defined as when a patient moves from above caseness to below caseness. Caseness is defined as a patient exhibiting clinically significant anxiety symptoms (a GAD-7 score of 8 or more) or clinically significant depression symptoms (a PHQ-9 score of 10 or more). One or both of the foregoing may be met for caseness to be achieved.

The number of patient variables 102 may be one or more, for example one, two, three, four, five or more patient variables 102. Likewise the number of service variables 104 may be one or more, for example one, two, three, four, five or more service variables 104. In general, the greater the number of patient variables 102 and/or service variables 104 that are collected as inputs in the method 100, the more reliable the prediction of psychological therapy outcome 116.

Patient variables 102 and/or service variables 104 are included in a model to predict clinical (psychological therapy) outcome (e.g. recovery/improvement) for a patient. This prediction is made based on historical patient data relating the characteristics of the patients who have already been treated to their known outcome, with a different weight assigned to each variable depending on how much they are determined to influence outcomes (e.g. initial symptom severity may impact recovery more than other variables such as age). When a new patient presents to the service, their characteristics (patient and/or service variables) are compared to those of the historical cohort, to calculate an aggregate score that represents a prediction of psychological therapy outcome for that patient, for example the likelihood/probability of a positive clinical outcome (recovery or improvement) for that patient. For example, the patient age is compared to the age distribution of the historical patient cohort, to assess to which extent the patient age would contribute to a good or a poor clinical outcome (e.g. higher age above cohort mean would contribute to a higher likelihood of a good clinical outcome). All patient variables 102 and service variables 104 are assessed in the same way, and the algorithm produces an aggregate score 112 that takes all these individual comparisons into account, producing an overall prediction of psychological therapy outcome 116 for that patient.

The prediction of psychological therapy outcome 116 may be used by the method to implement a particular action, for example the assignment of a treatment protocol.

Optionally, the prediction of psychological therapy outcome 116 may be used to assign a treatment protocol 118. For example, the treatment protocol 118 may include the frequency of one-to-one or face-to-face meetings, the frequency of asynchronous messaging in between sessions, the potential need for psychotropic medication(s), or treatment by a particular therapist as part of the treatment (e.g. IECBT) protocol. For example, a patient for whom the prediction of psychological therapy outcome is good, i.e. with a high probability of recovery and/or improvement, may be assigned to a treatment protocol 118 with fewer one-to-one or face-to-face meetings, than for a patient for whom the prediction of psychological therapy outcome is poor, i.e. with a low probability of recovery and/or improvement. In this context, a prediction of ‘good’ psychological therapy outcome means above average, whereas a prediction of ‘poor’ psychological therapy outcome means below average. In other words, a patient for whom the method gives a prediction of above average psychological therapy outcome (i.e. high probability of recovery and/or improvement), may be assigned to a treatment protocol 118 with fewer or lower frequency of one-to-one or face-to-face meetings (therapy sessions) than for a patient for whom the method gives a prediction of below average psychological therapy outcome, (i.e. low probability of recovery and/or improvement). In that way, patients receive appropriate treatment, and the therapy provider does not incur unnecessary costs associated with the over-provision of treatment to patients who have a high likelihood of improvement following therapy.

The scale may simply be a range from 0 to 1, where 0 means no likelihood of recovery and/or improvement, and 1 means certainty of recovery and/or improvement, for that patient. On such a scale, an aggregate score value equal to or greater than 0.5 for a patient means that patient has an average or above average likelihood of recovery and/or improvement, i.e. the prediction of psychological therapy outcome produced is ‘good’. An aggregate score value less than 0.5 for a patient means that patient has a below average likelihood of recovery and/or improvement, i.e. the prediction of psychological therapy outcome is ‘poor’.

The threshold (or criterion) is determined in any suitable way so as to provide a meaningful separation of different predictions of psychological therapy outcome. For different levels of control, more or fewer thresholds/criteria may be defined as desired. Data from a cohort of patients of known outcome (e.g. recovery) may be used to set the threshold(s)/criteria; the threshold(s)/criteria may then be applied to a new patient from a matched cohort. Suitably, the threshold(s)/criteria may be determined using a cost/benefit analysis in cases where there is a difference in cost for one of the possible actions of the method. Thereby the threshold(s)/criteria may be set to maximize effectiveness for patients whilst minimizing costs to the therapy service. In the above example of a scale from 0 to 1 where 0.5 represents average likelihood of recovery and/or improvement, the threshold/criteria may also be set to correlate with an aggregate score of 0.5. In that example all patients for whom the prediction of psychological outcome is ‘good’ (i.e. those with a score of 0.5 or greater) may be allocated to one treatment protocol, whereas all patients for whom the prediction of psychological outcome is ‘poor’ (i.e. those with a score of less than 0.5) may be allocated to a different treatment protocol. An exemplary treatment protocol for patients for whom the prediction of psychological outcome is ‘good’ may include the provision of reading (self-help) materials and/or a low frequency/number of one-to-one therapy sessions (e.g. one per week for four weeks), whereas an exemplary treatment protocol for patients for whom the prediction of psychological outcome is ‘poor’ may include a high frequency/number of one-to-one therapy sessions (e.g. two per week for six weeks). Further thresholds/criteria may be set as appropriate, for example an aggregate score value equal to or less than 0.25 for a patient may mean that the patient has a ‘very poor’ prediction of psychological therapy outcome; for example such patients may be allocated to a third treatment protocol wherein experienced therapists provide a high frequency/number of one-to-one therapy sessions (e.g. two per week for six weeks), and additionally an indication of a potential need for psychotropic medication may be provided.

Examples of actions of the method may include but are not limited to: the output of a recommendation of, or initiation of, a particular treatment protocol for the patient, suitable treatment protocols may comprise a specified frequency of one-to-one or face-to-face meetings, or a specified frequency of asynchronous messaging; the output of a recommendation of, or allocation of, a particular therapist for the patient; the output of a recommendation of, or provision of, reading materials (e.g. self-help) materials to the patient; the output of a recommendation of, or an indication of a potential need for, one or more (psychotropic) medication(s) for the patient.

Thereby, a prediction of psychological therapy outcome as provided by the method may form part of a method of treating a patient that may comprise: obtaining data relating to one or more patient variables and/or one or more service variables for a patient suffering from a mental health disorder; attributing a score to the data for each of the patient variables and/or the service variables, wherein the scores are based on a correlation between historic cohort treatment outcomes and historic cohort data comprising cohort patient and/or service variables; combining the scores to calculate an aggregate score; comparing the aggregate score with a scale to produce a prediction of psychological therapy outcome for the patient; and treating the patient according to a treatment protocol determined based on a comparison of the prediction of psychological therapy outcome for the patient and one or more thresholds/criteria derived from the correlation between the historic cohort treatment outcomes and the historic cohort data.

Furthermore, if the methods of the invention result in a prediction of below average (psycho)therapy outcome for a patient, a further step of the method may include the determination of particular ‘risk variables’ for that patient. The method may then implement an action modified by the particular risk variable(s) identified. Exemplary risk variables may include the presence of a long-term physical condition (physical comorbidity), or being in the lowest quartile in terms of age. Suitable risk variables may be determined from comparison with historic cohort data. In the example of a patient with lower than average likelihood of recovery and a long term physical condition, they may be assigned to a specific treatment protocol targeting psychological issues in the context of physical conditions. Thereby a tailored treatment protocol may be provided to the patient, increasing the likelihood of recovery by that patient, and minimizing costs to the therapy service.

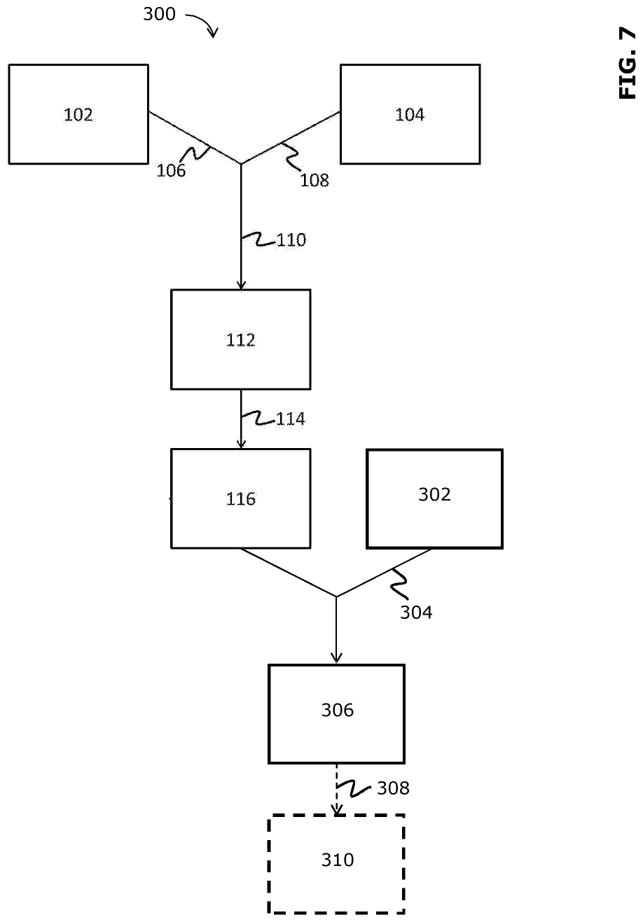

Referring to FIG. 7, a prediction of psychological therapy outcome as provided by the method may also form part of a method 300 of determining the effectiveness of a therapist, comprising: obtaining data relating to one or more patient variables 102 and/or one or more service variables 104 for one or more patient suffering from a mental health disorder and allocated to the therapist; attributing a score 106,108 to the data for each of the patient variables and/or the service variables for each patient, wherein the scores are based on a correlation between historic cohort treatment outcomes and historic cohort data comprising cohort patient and/or service variables; combining 110 the scores to calculate an aggregate score 112 for each patient; comparing the aggregate score with a scale 114 to produce a prediction of psychological therapy outcome 116 for the one or more patient; obtaining an observation of psychological therapy outcome 302 for the one or more patient after treatment by the therapist has been provided; and comparing 304 the observation of psychological therapy outcome and the prediction of psychological therapy outcome for the one or more patient to make a determination of the effectiveness of the therapist 306. Where data from more than one patient is used in the method of determining the effectiveness of a therapist, the data may be pooled at various points in the method. For example, the prediction of psychological therapy outcome for more than one patient may be meaned (to produce an ‘expected overall recovery rate’ (ORR)), and the observations of psychological therapy outcome for those same patients may be meaned (to produce an observed expected recovery rate (ORR)), and then the expected ORR is subtracted from the observed ORR to give a determination of the effectiveness of the therapist. Alternatively, for each patient treated by a particular therapist the prediction of psychological therapy outcome for that patient is subtracted from the observation of psychological therapy outcome for that patient. The resulting values are meaned for each patient treated by that therapist to give the determination of the effectiveness of the therapist. Where the determination of the effectiveness of a therapist has a positive value this signifies that the performance of the therapist was better than expected; the therapist is therefore deemed ‘most effective’, where it has a negative value this signifies that the performance of the therapist was worse than expected; the therapist is therefore deemed ‘least effective’. Where the determination of the effectiveness of a therapist equals zero, this means the therapist performed exactly as predicted by the method of the invention.

The determination of the effectiveness of a therapist may be used as the basis to take 308 one or more action 310. Suitable actions may include but are not limited to: (1) providing additional training materials to the therapist, (2) initiating additional supervision of the therapist, (3) initiating further training of the therapist, and (4) reallocating patients from the therapist to one or more other therapist. For example, where the determination of the effectiveness of the therapist has a negative value (the therapist performed worse than expected), any of the above actions may be taken in order to either improve the performance of the therapist in the future (providing additional training materials to the therapist; initiating further training of the therapist) or mitigate the potentially negative effects of a poorly performing therapist on future patients (initiating additional supervision of the therapist; reallocating patients from the therapist to one or more other therapist). Thereby the quality of the therapy provided to patients improves over time, benefitting patients, therapists, the therapy service and the payors of the therapy service.

The method of determining the effectiveness of a therapist may be provided by means of a non-transitory, tangible, computer-readable storage medium containing a program of instructions that cause a computer system running the program of instructions to carry out the method. The computer system may comprises one or more mobile device. Where the data received by the method is from more than one hardware source, such hardware sources may comprise one or more mobile device.

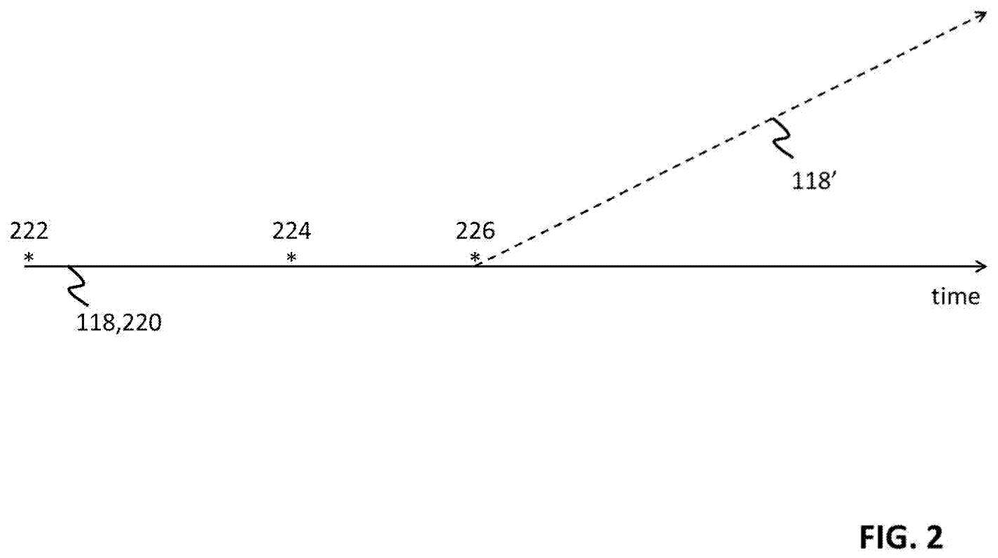

FIG. 2 illustrates a timeline 220 of the treatment protocol 118. With continued reference to FIG. 1 also, the computer-implemented method 100 of the present disclosure may be implemented at one or more points along the timeline 200. For example, the computer-implemented method 100 may be implemented before a treatment protocol 118 is implemented (e.g., at an initial assessment 222). Alternatively or additionally, the computer-implemented method 100 may be implemented during the treatment protocol 118 (e.g., at time point 224 and/or time point 226). During these later time points 224,226, the patient variables 102 (if available) may be updated as inputs, and the service variables 104 relative to the current treatment protocol 118 may be used as input. Then, the computer-implemented method 100 of the present disclosure may provide the prediction of psychological therapy outcome 116 relative to the current treatment protocol 118. In some instances, if needed, the treatment protocol 118 may change to a second treatment protocol 118′ optionally determined by the method 100 of the present disclosure.

The computer-implemented method 100 may continue being implemented to monitor the progress of the patient along either treatment protocol 118,118′. For example, in some instances, the prediction of psychological therapy outcome 116 may be computed two or more times (including initially and/or during treatment) where a comparison 228 of the prediction of psychological therapy outcome 116 at the different times can be used as a measure of the quality of a psychological therapy 230.

Referring again to FIG. 1, in some instances, when a fee-for-value payment system is utilized, the quality of the psychological therapy 230 may be used to determine the reimbursements 232 associated with the patient's care.

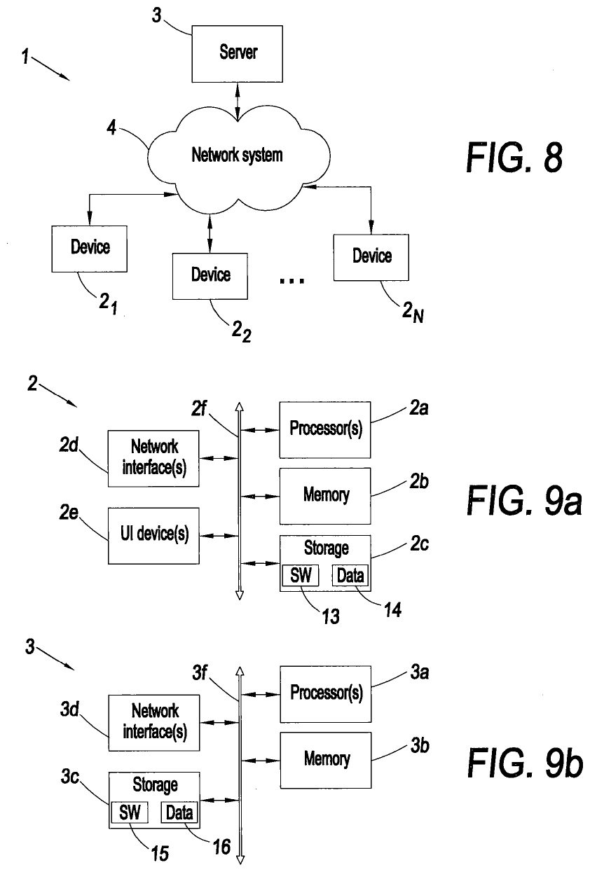

Systems and corresponding computer hardware used to implement the various illustrative blocks, modules, elements, components, methods, and algorithms relative to the methods 100,300 described herein can include a processor configured to execute one or more sequences of instructions, programming stances, or code stored on a non-transitory, computer-readable medium. The processor can be, for example, a general purpose microprocessor, a microcontroller, a digital signal processor, an application specific integrated circuit, a field programmable gate array, a programmable logic device, a controller, a state machine, a gated logic, discrete hardware components, an artificial neural network, or any like suitable entity that can perform calculations or other manipulations of data. In some embodiments, computer hardware can further include elements such as, for example, a memory (e.g., random access memory (RAM), flash memory, read only memory (ROM), programmable read only memory (PROM), erasable read only memory (EPROM)), registers, hard disks, removable disks, CD-ROMS, DVDs, or any other like suitable storage device or medium. In some embodiments of any aspect of the invention, the systems may comprise distributed systems, where the various illustrative blocks, modules, elements, components, methods, and algorithms relative to the methods 100 described herein may be performed on distributed computers/computing devices/hardware sources. Exemplary distributed computers/computing devices/hardware sources may be mobile or portable devices, for example tablet computers, smartphones, smart watches, laptops etc., therefore the methods 100 described herein may be at least partially performed on remote devices such as tablet computers, smartphones, smart watches, laptops etc. Distributed systems may also comprise central computer servers, and may make use of a cloud-computing approach, therefore the methods 100 described herein may be at least partially performed on central computer servers, and may make use of cloud computing. “Computer” as used herein may be understood to encompass a distributed computing system (e.g. one or more networked computers/devices/hardware sources), which may further encompass a cloud computing system. Referring to FIG. 8, a computer-based system 1 for performing the computer-implemented methods of the present invention includes a plurality of devices 2₁ . . . 2_{N} connectable to a server 3 via a network system 4. Each device 2 may be a mobile device, such as a laptop, tablet, smartphone, smart device, smart speaker, wearable device, etc. Each device 2 may be a (nominally) non-mobile device, such as desktop computer, etc. Each device 2 may be of any suitable type, such as a ubiquitous computing device, etc.

Referring to FIG. 9a, a (typical) device 2 includes one or more processors 2a, memory 2b, storage 2c, one or more network interfaces 2d, and one or more user interface (UI) devices 2e. The one or more processors 2a communicate with other elements of the device 2 via one or more buses 2f, either directly or via one or more interfaces (not shown). The memory 2b includes volatile memory such as dynamic random-access memory. Among other things, the volatile memory is used by the one or more processors 2a for temporary data storage, e.g. when controlling the operation of other elements of the device 2 or when moving data between elements of the device 2. The memory 2b includes non-volatile memory such as flash memory. Among other things, the non-volatile memory may store a basic input/output system (BIOS). The storage 2c includes e.g. solid-state storage and/or one or more hard disk drives. The storage 2c stores computer-readable instructions (SW) 13. The computer-readable instructions 13 include system software and application software. The application software preferably includes a web browser software application (hereinafter referred to simply as a web browser) among other things. The storage 2c also stores data 14 for use by the device 2. The one or more network interfaces 2d communicate with one or more types of network, for example an Ethernet network, a wireless local area network, a mobile/cellular data network, etc. The one or more user interface devices 2e preferably include a display and may include other output devices such as loudspeakers. The one or more user interface devices 2e preferably include a keyboard, pointing device (e.g. mouse) and/or a touchscreen, and may include other input device such as microphones, sensors, etc. Hence the device 2 is able to provide a user interface for e.g. a patient or therapist.

Referring to FIG. 9b, a (typical) server 3 may include one or more processors 3a, memory 3b, storage 3c, one or more network interfaces 3d, and one or more buses 3f. The elements of the server 3 are similar to the corresponding elements of the abovedescribed device 2. The storage 3c stores computer-readable instructions (SW) 15 (including system software and application software) and data 16 associated with the server 3. The application software preferably includes a web server among other things. The server 3 may be different from the abovedescribed server 3. For example, the server 3 may correspond to a virtual machine, a part of a cloud computing system, a computer cluster, etc.

Referring again to FIG. 8, the network system 4 preferably includes a plurality of networks, including one or more local area networks (e.g. Ethernet networks, Wi-Fi networks), one or more mobile/cellular data networks (e.g. 2nd, 3rd, 4th generation networks) and the Internet. Each device 2 is connectable to the server 3 via at least a part of the network system 4. Hence each device 2 is able to send and receive data (e.g. data constituting messages) to and from the server 3.

Executable sequences described herein can be implemented with one or more sequences of code (e.g., a set of instructions for implementing one or more methods 100 of the present disclosure) contained in a memory. In some embodiments, such code can be read into the memory from another machine-readable medium. Execution of the sequences of instructions contained in the memory can cause a processor to perform the process steps described herein. One or more processors in a multi-processing arrangement can also be employed to execute instruction sequences in the memory. In addition, hard-wired circuitry can be used in place of or in combination with software instructions to implement various embodiments described herein. Thus, the present embodiments are not limited to any specific combination of hardware and/or software.

As used herein, a machine-readable medium will refer to any medium that directly or indirectly provides instructions to a processor for execution.

A machine-readable medium can take on many forms including, for example, non-volatile media, volatile media, and transmission media. Non-volatile media can include, for example, optical and magnetic disks. Volatile media can include, for example, dynamic memory. Transmission media can include, for example, coaxial cables, wire, fiber optics, and wires that form a bus. Common forms of machine-readable media can include, for example, floppy disks, flexible disks, hard disks, magnetic tapes, other like magnetic media, CD-ROMs, DVDs, other like optical media, punch cards, paper tapes and like physical media with patterned holes, RAM, ROM, PROM, EPROM and flash EPROM.

Preferably, in some instances, implementation of the methods 100 described herein may be via a system approach where one or more of the patient variables 102 and/or one or more of the service variables 104 are provided and/or updated by the patient, the service provider, or the like at a remote location (e.g., via a computer, smart phone, or other comparable device). The data may then be communicated to a central computer, which performs one or more of the analysis methods 100 described herein. In such instances, one or more of the patient variables 102 and/or one or more of the service variables 104 may also be provided and/or updated at the central computer. In this example, the received data is from more than one hardware source.

Alternatively, the patient variables 102 and service variables 104 may be input to a central computer that performs one or more of the analysis methods 100 described herein. It will be understood that ‘central computer’ as used herein may encompass more than one computer or processor operating in a network or cloud computing arrangement.

In some instances, a cloud computing approach may be used to implement the methods 100 described herein. In some instances the methods 100 described herein may be (at least partially) implemented using a web browser on the remote device(s), in other instances the methods 100 may be (at least partially) implemented using an app (application software) on the remote device(s).

Unless otherwise indicated, all numbers expressing quantities of for example, patient variables, service variables, aggregate score, and so forth used in the present specification and associated claims are to be understood as being modified in all instances by the term “about.” Accordingly, unless indicated to the contrary, the numerical parameters set forth in the following specification and attached claims are approximations that may vary depending upon the desired properties sought to be obtained by the embodiments of the present invention. At the very least, and not as an attempt to limit the application of the doctrine of equivalents to the scope of the claim, each numerical parameter should at least be construed in light of the number of reported significant digits and by applying ordinary rounding techniques.

One or more illustrative embodiments incorporating the invention embodiments disclosed herein are presented herein. Not all features of a physical implementation are described or shown in this application for the sake of clarity. It is understood that in the development of a physical embodiment incorporating the embodiments of the present invention, numerous implementation-specific decisions must be made to achieve the developer's goals, such as compliance with system-related, business-related, government-related and other constraints, which vary by implementation and from time to time. While a developer's efforts might be time-consuming, such efforts would be, nevertheless, a routine undertaking for those of ordinary skill the art and having benefit of this disclosure.

While compositions and methods are described herein in terms of “comprising” various components or steps, the compositions and methods can also “consist essentially of” or “consist of” the various components and steps.

In a first aspect, the invention provides a method of predicting psychological therapy outcome comprising: obtaining data relating to one or more patient variables and/or one or more service variables for a patient; attributing a score to the data for each of the patient variables and/or the service variables; combining the scores to calculate an aggregate score; and using the aggregate score to make a prediction of psychological therapy outcome.

In one embodiment in accordance with any aspect of the invention, the method is a computer-implemented method.

In one embodiment, the method further comprises: assigning a treatment protocol to the patient based on the prediction of psychological therapy outcome.

In another embodiment, the method further comprises: assessing a general anxiety disorder 7-item (GAD-7) score and/or a patient health questionnaire (PHQ-9) score before implementing the psychological therapy, wherein the GAD-7 score and/or the PHQ-9 score is included as one or more of the patient variables.

In one embodiment, the psychological therapy in accordance with the method of the invention comprises internet-enabled cognitive behavioural therapy.

In another embodiment, the patient is suffering from a mental health disorder, wherein the disorder optionally comprises a disorder selected from the group consisting of (1) depression, (2) mixed anxiety and depression, (3) generalized anxiety disorder, (4) social phobias, (5) panic disorder, (6) obsessive-compulsive disorder, (7) post-traumatic stress disorder, (8) agoraphobia, (9) specific phobias, and (10) another anxiety disorder.

In one embodiment, the one or more patient variables comprises a variable selected from the group consisting of (1) patient gender, (2) patient age, (3) whether or not the patient suffered from a long-term physical condition, (4) whether or not the patient was taking psychotropic medication at the start of treatment, (5) the initial symptom severity, (6) the mental health disorder the patient suffers from, (7) whether or not a patient is currently pregnant, or has been pregnant or given birth in the previous 12 months, and (8) patient employment status. Suitably, the one more or patient variables comprises a variable selected from the group consisting of (1) patient gender, (2) patient age, (3) whether or not the patient suffered from a long-term physical condition, (4) whether or not the patient was taking psychotropic medication at the start of treatment, and (5) the initial symptom severity.

In another embodiment, the one or more service variables comprises a variable selected from the group consisting of (1) waiting times between various stages in the patient journey, (2) treatment duration, (3) the number of scheduled appointments the patient fails to attend, (4) the therapist the patient is allocated to, and (5) the therapeutic protocol the patient receives. Suitably, the one or more service variables comprises a variable selected from the group consisting of (1) waiting times between various stages in the patient journey, (2) treatment duration, and (3) the number of scheduled appointments the patient fails to attend.

In one embodiment, the prediction of psychological therapy outcome is a measure of improvement and/or a measure of recovery.

In one embodiment in accordance with any aspect of the invention, the method further comprises performing the method at two times during the psychological therapy to attain a first prediction of psychological therapy outcome and a second prediction of psychological therapy outcome; comparing the first and second predictions of psychological therapy outcome; and calculating a measure of quality of the psychological therapy. Suitably, the method may further comprise calculating a reimbursement value to the psychological therapy in a fee-for-value payment system based on the measure of quality of the psychological therapy.

In another aspect, the invention provides a data processing apparatus/device/system comprising means for carrying out the steps of the method in accordance with any aspect or embodiment of the invention, optionally wherein the data processing apparatus/device/system comprises one or more mobile device.

In another aspect, the invention provides a computer program comprising instructions which, when the program is executed by a computer, cause the computer to carry out the steps of the method according to any aspect or embodiment of the invention.

In a further aspect, the invention provides a computer-readable storage medium comprising instructions which, when executed by a computer, cause the computer to carry out the steps of the method according to any aspect or embodiment of the invention.

In another aspect, the invention provides a non-transitory, tangible, computer-readable storage medium: containing a program of instructions that cause a computer system running the program of instructions to: receive data relating to one or more patient variables and/or one or more service variables for a patient; attribute a score to the data for each of the patient variables and/or the service variables; combine the scores to calculate an aggregate score; and compare the aggregate score with an outcome scale to make a prediction of psychological therapy outcome.

In one embodiment of the non-transitory, tangible, computer-readable storage medium, the program of instructions further cause the computer system running the program of instructions to: assign a treatment protocol to the patient based on the prediction of psychological therapy outcome.

In another embodiment of the transitory, tangible, computer-readable storage medium, the program of instructions further cause the computer system running the program of instructions to: perform the method at two times during the psychological therapy to attain a first prediction of psychological therapy outcome and a second prediction of psychological therapy outcome; compare the first and second predictions of psychological therapy outcome; and calculate a measure of quality of the psychological therapy. Suitably, the program of instructions may further cause the computer system running the program of instructions to calculate a reimbursement value to the psychological therapy in a fee-for-value payment system based on the measure of quality of the psychological therapy.

In another embodiment of the transitory, tangible, computer-readable storage medium, the received data is from more than one hardware source, optionally wherein the one or more hardware source comprises a mobile device.

In another aspect, the invention provides a computer-implemented method of providing psychological therapy to (treating) a patient, the method comprising: obtaining data relating to one or more patient variables and/or one or more service variables for a patient suffering from a mental health disorder; attributing a score to the data for each of the patient variables and/or the service variables, wherein the scores are based on a correlation between historic cohort treatment outcomes and historic cohort data comprising cohort patient and/or service variables; combining the scores to calculate an aggregate score; comparing the aggregate score with a scale to produce a prediction of psychological therapy outcome for the patient; and providing psychological therapy to (treating) the patient according to a treatment protocol determined based on a comparison of the prediction of psychological therapy outcome for the patient and one or more thresholds/criteria derived from the correlation between the historic cohort treatment outcomes and the historic cohort data.

In another aspect, the invention provides a data processing apparatus/device/system comprising means for carrying out the steps of the computer-implemented method of providing psychological therapy to (treating) a patient, optionally wherein the data processing apparatus/device/system comprises one or more mobile device.

In another aspect, the invention provides a computer program (product) comprising instructions which, when the program is executed by a computer, cause the computer to carry out the steps of the computer-implemented method of providing psychological therapy to (treating) a patient.

In a further aspect, the invention provides a computer-readable storage medium comprising instructions which, when executed by a computer, cause the computer to carry out the steps of the computer-implemented method of providing psychological therapy to (treating) a patient.

In another aspect, the invention provides a method of treating a patient, comprising: obtaining data relating to one or more patient variables and/or one or more service variables for a patient suffering from a mental health disorder; attributing a score to the data for each of the patient variables and/or the service variables, wherein the scores are based on a correlation between historic cohort treatment outcomes and historic cohort data comprising cohort patient and/or service variables; combining the scores to calculate an aggregate score; comparing the aggregate score with a scale to produce a prediction of psychological therapy outcome for the patient; and treating the patient according to a treatment protocol determined based on a comparison of the prediction of psychological therapy outcome for the patient and one or more thresholds/criteria derived from the correlation between the historic cohort treatment outcomes and the historic cohort data.

In one embodiment of the method of treating a patient, the treatment protocol comprises one or more treatments selected from the group consisting of: (1) a specified frequency of one-to-one or face-to-face meetings, (2) a specified frequency of asynchronous messaging, (3) the provision of self-help materials, (4) an indication of a potential need for psychotropic medication(s), and (5) the allocation of a particular therapist.

In another embodiment of the method of treating a patient, the method further comprises: assessing a general anxiety disorder 7-item (GAD-7) score and/or a patient health questionnaire (PHQ-9) score before implementing the psychological therapy, wherein the GAD-7 score and/or the PHQ-9 score is included as one or more of the patient variables.

In another embodiment of the method of treating a patient, the psychological therapy comprises internet-enabled cognitive behavioural therapy.

In another embodiment of the method of treating a patient, the mental health disorder comprises a disorder selected from the group consisting of (1) depression, (2) mixed anxiety and depression, (3) generalized anxiety disorder, (4) social phobias, (5) panic disorder, (6) obsessive-compulsive disorder, (7) post-traumatic stress disorder, (8) agoraphobia, (9) specific phobias, and (10) another anxiety disorder.

In another embodiment of the method of treating a patient, the one or more patient variables comprises a variable selected from the group consisting of (1) patient gender, (2) patient age, (3) whether or not the patient suffers from a long-term physical condition, (4) whether or not the patient is taking psychotropic medication at the start of treatment, (5) the initial symptom severity, (6) the mental health disorder the patient suffers from, (7) whether or not a patient is currently pregnant, or has been pregnant or given birth in the previous 12 months, and (8) patient employment status.

In another embodiment of the method of treating a patient, the one or more service variables comprises a variable selected from the group consisting of (1) waiting times between various stages in the patient journey, (2) treatment duration, (3) the number of scheduled appointments the patient fails to attend, (4) the therapist the patient is allocated to, and (5) the treatment protocol the patient receives.

In another embodiment of the method of treating a patient, the prediction of psychological therapy outcome for the patient is a measure of improvement and/or a measure of recovery.

In another embodiment of the method of treating a patient, the method further comprises performing the method at two times during the psychological therapy to attain a first prediction of psychological therapy outcome for the patient and a second prediction of psychological therapy outcome for the patient; comparing the first and second prediction of psychological therapy outcome for the patient; and using the comparison of first and second prediction of psychological therapy outcome to calculate a measure of quality of the psychological therapy. Suitably, the method of treating a patient may further comprise calculating a reimbursement value to the psychological therapy in a fee-for-value payment system based on the measure of quality of the psychological therapy.

In another embodiment of the method of treating a patient, the method further comprises obtaining second data relating to the one or more patient variables and/or the one or more service variables for the patient at a time after beginning the treatment protocol; attributing a second score to the second data for each of the patient variables and/or the service variables; combining the second scores to calculate a second aggregate score; using the second aggregate score to make a second prediction of psychological therapy outcome for the patient; and treating the patient according to a second treatment protocol determined based on the second prediction of psychological therapy outcome for the patient.

In one embodiment of the method of treating a patient, the method further comprises determining whether the patient suffers from a long-term physical condition; and treating the patient according to a treatment protocol determined based on both the prediction of psychological therapy outcome for the patient and the determination of whether the patient suffers from a long-term physical condition.

In another aspect, the invention provides a computer-implemented method of determining the effectiveness of a therapist, the method comprising obtaining data relating to one or more patient variables and/or one or more service variables for one or more patient suffering from a mental health disorder and allocated to the therapist; attributing a score to the data for each of the patient variables and/or the service variables, wherein the scores are based on a correlation between historic cohort treatment outcomes and historic cohort data comprising cohort patient and/or service variables; combining the scores to calculate an aggregate score; comparing the aggregate score with a scale to produce a prediction of psychological therapy outcome for the one or more patient; obtaining an observation of psychological therapy outcome for the one or more patient after treatment by the therapist has been provided; comparing the observation of psychological therapy outcome and the prediction of psychological therapy outcome for the one or more patient to make a determination of the effectiveness of the therapist.

In another aspect, the invention provides a data processing apparatus/device/system comprising means for carrying out the steps of the computer-implemented method of determining the effectiveness of a therapist, optionally wherein the data processing apparatus/device/system comprises one or more mobile device.

In another aspect, the invention provides a computer program (product) comprising instructions which, when the program is executed by a computer, cause the computer to carry out the steps of the computer-implemented method of determining the effectiveness of a therapist.

In a further aspect, the invention provides a computer-readable storage medium comprising instructions which, when executed by a computer, cause the computer to carry out the steps of the computer-implemented method of determining the effectiveness of a therapist.

In another aspect, the invention provides a method of determining the effectiveness of a therapist, comprising: obtaining data relating to one or more patient variables and/or one or more service variables for one or more patient suffering from a mental health disorder and allocated to the therapist; attributing a score to the data for each of the patient variables and/or the service variables, wherein the scores are based on a correlation between historic cohort treatment outcomes and historic cohort data comprising cohort patient and/or service variables; combining the scores to calculate an aggregate score; comparing the aggregate score with a scale to produce a prediction of psychological therapy outcome for the one or more patient; obtaining an observation of psychological therapy outcome for the one or more patient after treatment by the therapist has been provided; comparing the observation of psychological therapy outcome and the prediction of psychological therapy outcome for the one or more patient to make a determination of the effectiveness of the therapist.

In one embodiment of the method of determining the effectiveness of a therapist, the method further comprises taking an action based on the determination of the effectiveness of the therapist. Suitably, the action comprises an action selected from the group consisting of (1) providing additional training materials to the therapist, (2) initiating additional supervision of the therapist, (3) initiating further training of the therapist, (4) calculating a reimbursement value for the psychological therapy in a fee-for-value payment system, and (5) reallocating patients from the therapist to one or more other therapist.

In some embodiments of any aspect of the invention each step of the method may be performed in a step-wise manner. It will be understood by the person skilled in the art that in other embodiments of any aspect of the invention a number of steps of the method may be performed in any practical order. Alternatively, two or more steps may be conducted contemporaneously.

In another aspect, the invention provides a non-transitory, tangible, computer-readable storage medium: containing a program of instructions that cause a computer system running the program of instructions to: receive data relating to one or more patient variables and/or one or more service variables for one or more patient suffering from a mental health disorder and allocated to the therapist; attribute a score to the data for each of the patient variables and/or the service variables, wherein the scores are based on a correlation between historic cohort treatment outcomes and historic cohort data comprising cohort patient and/or service variables; combine the scores to calculate an aggregate score; compare the aggregate score with a scale to produce a prediction of psychological therapy outcome for the one or more patient; receive further data relating to an observation of psychological therapy outcome for the one or more patient after treatment by the therapist has been provided; compare the observation of psychological therapy outcome and the prediction of psychological therapy outcome for the one or more patient to make a determination of the effectiveness of the therapist.

In one embodiment of the non-transitory, tangible, computer-readable storage medium, the program of instructions further cause the computer system running the program of instructions to: take an action based on the determination of the effectiveness of the therapist. Suitably, the action comprises an selected from the group consisting of (1) providing additional training materials to the therapist, (2) initiating additional supervision of the therapist, (3) initiating further training of the therapist, (4) reallocating patients from the therapist to one or more other therapist and (5) calculating a reimbursement value for the psychological therapy in a fee-for-value payment system.

In one embodiment of the non-transitory, tangible, computer-readable storage medium, the received data is from more than one hardware source, optionally wherein the hardware source comprises a mobile device.

To facilitate a better understanding of the embodiments of the present invention, the following examples of preferred or representative embodiments are given. In no way should the following examples be read to limit, or to define, the scope of the invention.

### EXAMPLES

### Example 1

Methods: Data was analyzed from patients receiving either IECBT or standard care for the treatment of a mental health disorder, between April 2015 and March 2016.

IECBT was delivered to 4,468 patients using a commercial package, originally developed for and currently used in the English National Health Service, provided by Ieso Digital Health. Patients self-referred or were referred by a primary healthcare worker directly to the service. NICE approved CBT therapy, based on Beckian models and Roth and Pilling's CBT competences framework, was delivered in a secure online therapy room via instant synchronous messaging, by a qualified CBT therapist accredited by the British Association for Behavioral & Cognitive Psychotherapies (BABCP). As part of the IECBT therapy, in between appointments, asynchronous messages and homework tasks are exchanged between therapist and patient, promoting engagement and adherence to evidence-based treatment models.

A reference group receiving standard care consisted of 1,299,525 patients referred to IAPT services in England for the treatment of a mental health disorder. This includes both face-to-face and online therapy services. The standard care group data was extracted from the IAPT annual report for 2015/2016. Details of both cohorts can be found in Table 1.

TABLE 1
Demographic and severity details for the two patient cohorts,
for patients finishing a course of treatment between April 2015
and March 2016. The Internet-enabled CBT cohort was severity matched
to the standard care cohort using a multivariate rejection sampling.
The term “SD” below refers to standard deviation.

| | Internet-enabled CBT | | | |
|---|---|---|---|---|
| | (Severity matched) | | Standard care | |
| | (N = 2,207) | | (N = 534,259) | |
| | Mean/Prevalence | SD | Mean/Prevalence | SD |
| PHQ-9 at assessment | 14.8 | 3.1 | 14.9 | 3.2 |
| GAD-7 at assessment | 13.5 | 2.7 | 13.5 | 2.9 |
| Gender | 75% Female | — | 65% Female | — |
| Long-Term Conditions | 22% | — | 21% | — |
| Psychotropic Medication at assessment | | | | |
| Prescribed Not Taking | 8% | — | 5% | — |
| Prescribed Taking | 42% | — | 45% | — |
| Not Prescribed | 49% | — | 37% | — |
| Disorder | | | | |
| Depression | 29% | — | 30% | — |
| Mixed anxiety and depression | 23% | — | 29% | — |
| Generalized anxiety disorder | 11% | — | 15% | — |
| Social phobias | 6% | — | 2% | — |
| Panic disorder | 4% | — | 3% | — |
| Obsessive-compulsive disorder | 2% | — | 2% | — |
| Post-traumatic stress disorder | 3% | — | 3% | — |
| Agoraphobia | 1% | — | 1% | — |
| Specific phobias | 1% | — | 1% | — |
| Other anxiety disorder | 11% | — | 3% | — |
| Other mental health disorder | 11% | — | 10% | — |

The information captured through IAPT's minimum dataset intends to support monitoring of implementation and effectiveness of national policy/legislation, policy development, performance analysis and benchmarking, national analysis and statistics, and national audit of IAPT services. At registration, patients agree to the services' terms and conditions, including use of anonymized data to support research, including academic publications or conference presentations.

Clinical effectiveness analyses: For treatment effectiveness analyses, the two groups were compared for clinical outcomes measured in terms of treatment engagement, clinical recovery and improvement. Engagement is a binary measure (i.e. 0 or 1) of whether or not a patient engages with treatment. A patient was classed as engaged if they attended two or more therapy sessions. The engagement rate for each group was calculated as number of patients engaged, divided by total number of patients discharged in the given time period.

Clinical recovery and improvement were calculated based on two severity measures completed by the patient at initial assessment and for every therapy session (completion rate 95%): GAD-7 and PHQ-9, corresponding to anxiety and depressive symptoms respectively.

The GAD-7 is a seven-item screening and severity measure for generalized anxiety disorder. Under IAPT guidance, a patient scoring 8 or more in the GAD-7 (range 0-21) is considered to be suffering from clinically significant anxiety symptoms. This is referred to as meeting caseness. A reduction of 4 points or more on the GAD-7 scale between two time points is indicative of statistically reliable improvement in symptom severity. The PHQ-9 is a nine-item measure designed to facilitate screening and severity assessment of depression. Under IAPT guidance a patient scoring 10 or more in the PHQ-9 (range 0-27) is considered to be suffering from clinically significant depressive symptoms. A reduction of 6 points or more on the PHQ-9 scale between two time points is indicative of statistically reliable improvement in symptom severity. If a patient scored above caseness for one or both of these measures at initial assessment (i.e., 8 or above for GAD-7, and/or 10 or above for PHQ-9), they were classed as meeting caseness at assessment. Other symptom severity measures, such as severity scores for subtypes of anxiety disorders, were not examined as only GAD-7 and PHQ-9 are mandatorily collected within the IAPT framework.

For engaged patients, the difference between scores at initial assessment and last treatment session for GAD-7 and PHQ-9 was used to determine patients' recovery status. Recovery is a binary measure. Engaged patients who moved from above caseness at assessment to below caseness at the last treatment session were classed as recovered. Under the American healthcare system, this is more often known as remission. The recovery rate for each group was calculated as number of patients recovered, divided by number of patients at caseness at initial assessment.

Improvement is also a binary measure. Engaged patients who showed a significant reduction in at least one of the outcome measures (i.e. decrease of 4 points or more in the GAD-7 and/or 6 points or more in the PHQ-9) from assessment to the last treatment session, whilst not showing a significant increase in the other outcome measure, were classed as improved. The improvement rate for each group was calculated as number of patients improved, divided by number of engaged patients.

Clinical effectiveness analyses were performed in SPSS (IBM SPSS Statistics version 24) by comparing number of patients engaged, recovered and improved for both groups, using Pearson's chi-square test with Yates' continuity correction. For clinical effectiveness analyses comparing recovery and improvement rates, engaged patients in the IECBT group (N=2,207) were matched to the reference group for severity (PHQ-9 and GAD-7 scores at assessment), using a multivariate rejection sampling algorithm implemented in R (Table 1).

Regression analyses—predictors of clinical response in IECBT: Logistic regression analyses were performed in R to identify significant predictors of recovery and improvement in patients receiving IECBT, based on patient demographics and service variables described above.

Given the nature of the statistical models employed, record sets were included only for cases with complete data for all predictor variables. Of the initial sample of 4,468 patients, 2,211 engaged in treatment and 1,818 were at caseness at assessment and therefore evaluable. Of the evaluable record sets, 95% had complete data for all predictor variables and were included in the analyses (N=2,104 for improvement analysis, N=1,728 for recovery analysis).

Continuous predictor variables were scaled and centered to the mean. Multicollinearity analyses were performed to investigate potential correlations between predictor variables. Statistical significance was defined as P<0.05 two-tailed, uncorrected.

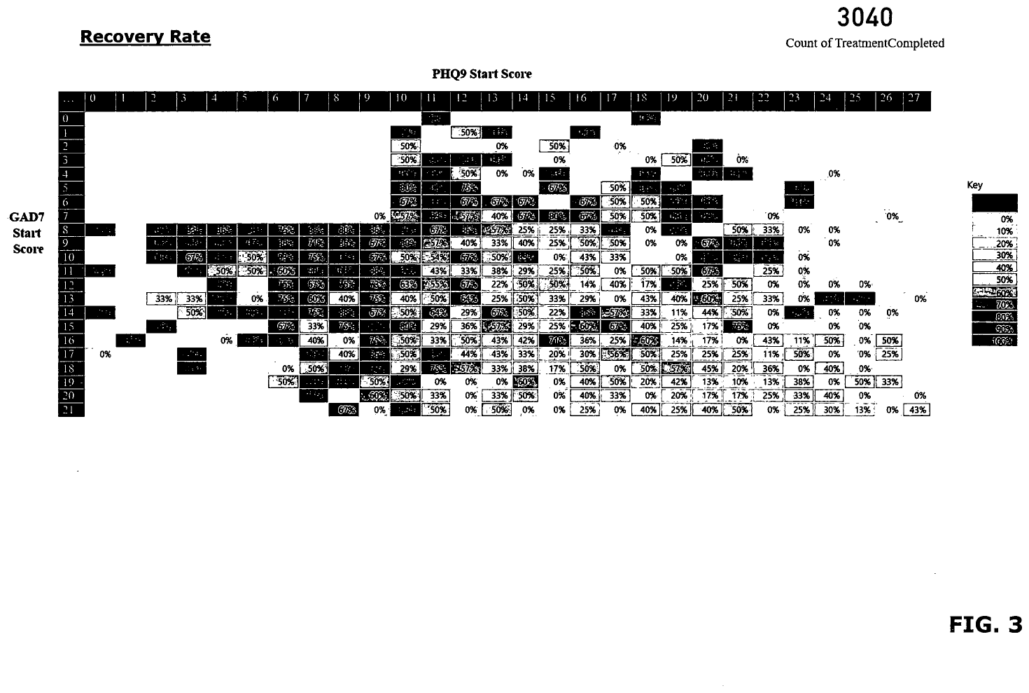

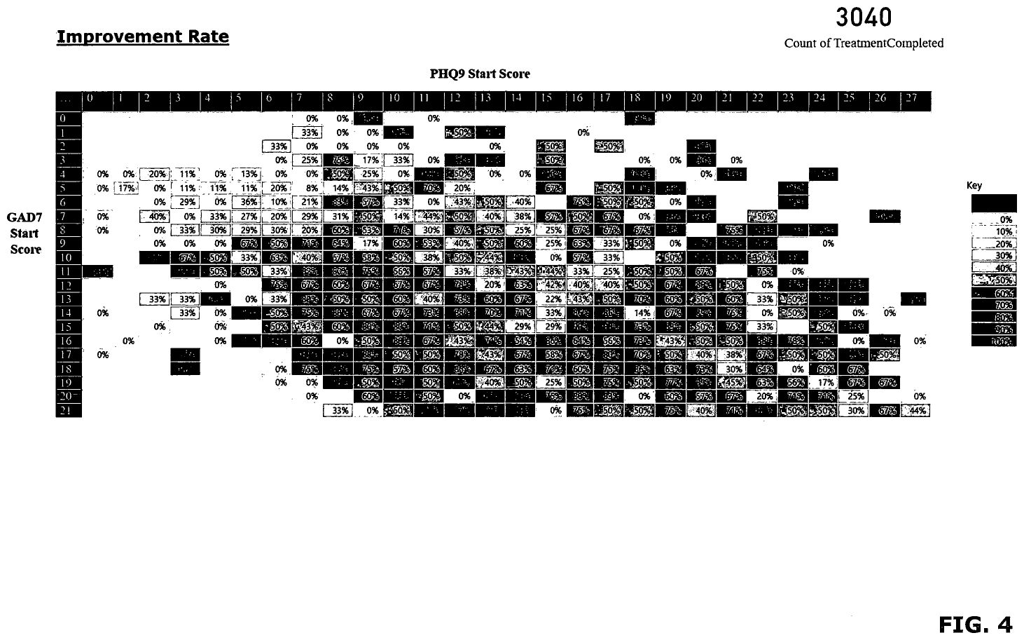

Clinical effectiveness analyses: Variations were observed in the likelihood of improvement and recovery with PHQ-9 and GAD-7 scores at assessment, for patients treated with IECBT (FIGS. 3 and 4). More severe patients, with higher PHQ-9 and GAD-7 scores at assessment, have lower likelihoods of recovery but equivalent or higher likelihood for improvement (FIGS. 3 and 4). Accordingly, comparative analyses of clinical improvement and recovery were conducted on severity-matched cohorts.

The improvement rate for severity-matched patients treated with IECBT was significantly higher than for patients treated with standard care (IECBT/Standard care: 67.5%/62.2% improvement rate, odds ratio=1.26, X²(1)=26.40, P<0.001).

Regarding clinical recovery no significant differences were observed between the two groups (IECBT/Standard care: 47.5%/46.5% recovery rate, odds ratio=1.04, X²(1)=0.85, P=0.356).

IECBT was also associated with significantly higher engagement rates compared to standard care (IECBT/Standard care: 49.5%/41.3% engagement rate, odds ratio=1.39, X²(1)=121.68, P<0.001).

Regression analyses—predictors of clinical response in IECBT: Logistic regression analyses identified the presence of long-term conditions, initial GAD-7 scores, waiting time for assessment, total number of treatment sessions and patient age as significant predictors of improvement (Table 2). Apart from waiting time for assessment, these variables were also identified as significant predictors of recovery, in addition to initial PHQ-9 scores (Table 3).

TABLE 2
Results of logistic regression analysis investigating predictors
of improvement in the Internet-enabled CBT cohort. Gender “Male”,
Long-Term Conditions “No” and Psychotropic Medication
“Prescribed Not Taking” were reference classes for the categorical
variables. The terms in the Table below are used as follows: StartPhq9 -
PHQ-9 score at assessment; StartGad7 - GAD-7 score at assessment; Waiting
SAQ - time between referral and patient completing self-assessment
questionnaire; WaitingAssignment - waiting time from patient completing
the self-assessment questionnaire and therapist assignment; WaitingContact -
waiting time between therapist assignment and first contact by the therapist;
WaitingAssessment - waiting time between first contact from the therapist
and clinical assessment appointment; WaitingTreatment - waiting time between
clinical assessment and first therapy session; NumberSessions - total number
of therapy sessions attended by the patient; NumberDNA - number of scheduled
appointments the patient failed to attend.
Predictors of Improvement in Internet-enabled CBT (N = 2,104)

| | | | | | Wald's | |
|---|---|---|---|---|---|---|
| | | | | | statistic | |
| Predictor variable | Mean/Prevalence | | B | SE | (z²) | P-value |
| Gender | | | | | | |
| Male | 27.3% | | — | — | — | — |
| Female | 72.0% | | 0.04 | 0.11 | 0.17 | .679 |
| Unknown/Not stated | 0.6% | | −0.24 | 0.59 | 0.16 | .687 |
| Age | 36.8 | years | 0.12 | 0.05 | 5.96 | .015 * |
| Long-Term Conditions | | | | | | |
| No | 34.2% | | — | — | — | — |
| Yes | 20.8% | | −0.32 | 0.14 | 5.62 | .018 * |
| Unknown/Not stated | 44.9% | | 0.03 | 0.11 | 0.06 | .813 |
| Psychotropic Medication | | | | | | |
| Prescribed Not Taking | 8.1% | | — | — | — | — |
| Prescribed Taking | 38.4% | | 0.24 | 0.18 | 1.73 | .189 |
| Not Prescribed | 52.4% | | 0.26 | 0.18 | 2.16 | .142 |
| Unknown/Not stated | 1.0% | | 0.22 | 0.49 | 0.20 | .651 |
| StartPhq9 | 12.7 | | 0.06 | 0.06 | 1.02 | .312 |
| StartGad7 | 11.9 | | 0.52 | 0.06 | 74.84 | <.001 *** |
| WaitingSAQ | 3.1 | days | 0.01 | 0.06 | 0.01 | .917 |
| WaitingAssignment | 0.8 | days | −0.04 | 0.06 | 0.41 | .523 |
| WaitingContact | 1.1 | days | −0.08 | 0.08 | 1.04 | .309 |
| WaitingAssessment | 7.6 | days | −0.14 | 0.05 | 7.70 | .006 ** |
| WaitingTreatment | 8.9 | days | −0.02 | 0.05 | 0.14 | .712 |
| NumberSessions | 5.5 | sessions | 0.29 | 0.05 | 32.33 | <.001 *** |
| NumberDNA | 0.5 | sessions | −0.01 | 0.05 | 0.1 | .776 |

TABLE 3
Results of logistic regression analysis investigating predictors
of recovery in the Internet-enabled CBT cohort. Gender “Male”,
Long-Term Conditions “No” and Psychotropic Medication
“Prescribed Not Taking” were reference classes for the
categorical variables. The terms in the Table below as used as follows:
StartPhq9 - PHQ-9 score at assessment; StartGad7 - GAD-7 score at
assessment; Waiting SAQ - time between referral and patient completing
self-assessment questionnaire; WaitingAssignment - waiting time from
patient completing the self-assessment questionnaire and therapist
assignment; WaitingContact - waiting time between therapist assignment
and first contact by the therapist; WaitingAssessment - waiting time
between first contact from the therapist and clinical assessment
appointment; WaitingTreatment - waiting time between clinical assessment
and first therapy session; NumberSessions - total number of therapy
sessions attended by the patient; NumberDNA - number of scheduled
appointments the patient failed to attend.
Predictors of Recovery in Internet-enabled CBT (N = 1,728)

| | | | | | Wald's | |
|---|---|---|---|---|---|---|
| | | | | | statistic | |
| Predictor variable | Mean/Prevalence | | b | SE | (z²) | P-value |
| Gender | | | | | | |
| Male | 26.6% | | — | — | — | — |
| Female | 72.9% | | 0.15 | 0.12 | 1.54 | .214 |
| Unknown/Not stated | 0.5% | | −0.14 | 0.70 | 0.04 | .841 |
| Age | 36.3 | years | 0.23 | 0.05 | 17.89 | <.001 *** |
| Long-Term Conditions | | | | | | |
| No | 33.2% | | — | — | — | — |
| Yes | 22.0% | | −0.36 | 0.15 | 5.73 | .017 * |
| Unknown/Not stated | 44.8% | | −0.13 | 0.12 | 1.25 | .263 |
| Psychotropic Medication | | | | | | |
| Prescribed Not Taking | 8.7% | | — | — | — | — |
| Prescribed Taking | 41.8% | | 0.02 | 0.20 | 0.01 | .921 |
| Not Prescribed | 48.3% | | 0.06 | 0.19 | 0.09 | .767 |
| Unknown/Not stated | 1.2% | | −0.49 | 0.53 | 0.85 | .357 |
| StartPhq9 | 14.3 | | −0.56 | 0.06 | 85.06 | <.001 *** |
| StartGad7 | 13.4 | | −0.30 | 0.06 | 26.97 | <.001 *** |
| WaitingSAQ | 3.0 | days | 0.01 | 0.06 | 0.01 | .933 |
| WaitingAssignment | 0.8 | days | 0.04 | 0.06 | 0.36 | .548 |
| WaitingContact | 1.2 | days | −0.11 | 0.07 | 2.17 | .141 |
| WaitingAssessment | 7.4 | days | −0.08 | 0.06 | 2.08 | .149 |
| WaitingTreatment | 8.8 | days | −0.02 | 0.05 | 0.19 | .667 |
| NumberSessions | 5.6 | sessions | 0.32 | 0.05 | 33.77 | <.001 *** |
| NumberDNA | 0.5 | sessions | −0.11 | 0.05 | 3.81 | .051 |

Results showed that patients with long-term physical conditions are less likely to show good clinical outcomes compared to patients without long-term conditions (Improvement: b=−0.32, SE=0.14, Wald z²=5.62, P=0.018; Recovery: b=−0.36, SE=0.15, Wald z²=5.73, P=0.017). Patients with higher severity scores at assessment were also less likely to show clinical recovery (StartPhq9Score: b=−0.56, SE=0.06, Wald z²=85.06, P<0.001; StartGad7Score: b=−0.30, SE=0.06, Wald z²=26.97, P<0.001). However, in line with what can be observed from FIG. 3, results suggested that patients with higher GAD-7 scores at assessment have higher likelihood of showing clinical improvement (b=0.52, SE=0.06, Wald z²=74.84, P<0.001).

A significant positive association between patient age and likelihood of good clinical outcomes was also observed (Improvement: b=0.12, SE=0.05, Wald z²=5.96, P=0.015; Recovery: b=0.23, SE=0.05, Wald z²=17.89, P<0.001). Additionally, results showed that patients who have undergone a larger number of therapy sessions were more likely to show good clinical outcomes (Improvement: b=0.29, SE=0.05, Wald z²=32.33, P<0.001; Recovery: b=0.32, SE=0.05, Wald z²=33.77, P<0.001) (Tables 2 and 3).

Tests of the full models against constant only models were significant for both regression analyses (Improvement regression model: X²(17)=194.82, P<0.001; Recovery regression model: X²(17)=278.54, P<0.001). Additionally, multicollinearity analyses revealed variance inflation factors smaller than 2 for all predictor variables, confirming that regression models were not affected by the presence of multicollinearity.

This study supports IECBT as a clinically effective therapy for patients with common mental health disorders. In severity matched cohorts, significantly higher improvement rates were observed for IECBT relative to standard care (67.5% vs 62.2%), with no significant difference observed in recovery rates (47.5% vs 46.5%).

Despite strong evidence supporting the clinical effectiveness of online therapy, previous research also showed that these modalities are often associated with high dropout rates. Contrary to this, the current study showed significantly higher engagement rates in IECBT relative to standard care, including both self-help and face-to-face services (49.5% vs 41.3%). The relatively high IECBT engagement rate observed in the present study is consistent with prior research showing lower dropout rates for therapist-guided online therapy compared to self-guided online programs.

Past research also showed that online CBT facilitates patient disclosure, with patients being more comfortable in confiding sensitive information through a computer rather than face-to-face. It is possible that IECBT eases patient disclosure, cementing the therapeutic relationship at an early stage, encouraging engagement and leading to better clinical outcomes. Furthermore, the present IECBT model allows therapist and patient to communicate via asynchronous messaging in between sessions, encouraging homework completion and adherence to evidence-based treatment protocols. This additional communication between therapist and patient, which is not available for standard care, may also contribute to engagement and good clinical outcomes.

Regarding predictions of clinical outcomes, analyses revealed a significant association between initial psychometric scores and likelihood of recovery, with higher scores associated with lower recovery rates, as illustrated in FIG. 4. By definition, patients recover by going below the caseness threshold for both PHQ-9 and GAD-7; therefore, it is not unexpected that patients whose initial scores are closer to that threshold have higher chances of recovery. This does however raise the question of whether recovery is a suitable index to measure clinical outcome. As indicated by Gyani and colleagues the recovery metric does not take into account whether the observed reduction in severity is greater than the measurement error of the scales. Conversely, the improvement index is a measure of whether or not a reduction in severity is statistically reliable, regardless of caseness, and may therefore be a better metric for widespread use. In this case, patients with higher initial scores are more likely to show clinical improvement, as validated by the results of the regression analysis, where patients with higher initial GAD-7 scores showed higher likelihoods of improvement (FIG. 3 and Table 2). Whilst differences in recovery rate with severity may be expected in this context, they may also indicate the presence of nonspecific treatment effects. Future strategies to improve treatment effectiveness are therefore likely to involve the implementation of measures aimed at boosting recovery of more severe patients and homogenizing recovery rates across the severity spectrum, such as increased session frequency at the start of treatment.

Regression analyses on improvement and recovery also revealed significant associations between clinical outcomes and age, presence of a long-term physical condition and number of therapy sessions. Results show that increasing age is associated with better clinical outcomes, contradicting previous research showing lower effectiveness of CBT in older adults. However, post-hoc analyses revealed a significant negative correlation between patient age and symptom severity (Age and PHQ-9: r=−0.08, t=−4.10, df=2416, P<0.001; Age and GAD-7: r=−0.13, t=−6.29, df=2416, P<0.001), suggesting the observed association between age and clinical outcomes may be explained by decreasing severity with age in this particular cohort.

In this cohort, it was also observed that patients with long-term physical conditions were less likely to show good clinical outcomes. This finding is unsurprising given that long-term physical conditions are often associated with comorbid mental health problems, which may themselves be chronic in nature and often treatment resistant. Lower probability of response to treatment may signal the need for tailored, condition-specific CBT models, so patients can be helped to deal with mental and physical symptoms in an integrated fashion. However, it could also be argued that PHQ-9 and GAD-7, used to calculate clinical outcomes, lack sensitivity to detect clinically significant improvements in patients with long-term conditions. Disease/disorder specific measures may provide better indicators of clinical improvement in these cases and could optionally be included as patient variables.

Service variables shown to be associated with the likelihood of good clinical outcomes included number of therapy sessions and waiting time for assessment, although care should be taken when interpreting cause-effect relationships. At first glance these results convey the impression that longer courses of treatment are associated with better clinical outcomes. However, an alternative explanation is that patients who do not adhere to their treatment plan and drop-out before completing a full course of treatment, therefore receiving a sub-therapeutic dose, are less likely to achieve good clinical outcomes. Additionally, patients who take longer to schedule their clinical assessment may be less motivated to engage in treatment, more likely to drop-out and therefore achieve poorer clinical outcomes.

Overall the results of the current study demonstrate that IECBT delivers clinical outcomes that are significantly different to standard care, with higher improvement and engagement rates. However, it is important to note that due to the nature of the aggregated audit reference data for standard care it was not possible to control for variables such as therapy type in the analysis. Whilst all patients in the IECBT group received CBT, patients in the standard care group received a range of different therapy types, with only a third of patients receiving CBT. It can be hypothesized that differences in therapy type, together with potential cohort differences in other uncontrolled variables such as IQ and socio-economic status, may also account for variance in engagement and clinical outcomes. Additionally, due to the focus of the analysis and the aggregated nature of the reference data, in this study data was pooled for all clinical populations. With CBT/IECBT response rates likely to vary between conditions, future analysis of this or additional data sets could optionally focus on individual disorders, for example but not limited to those listed in Table 1, using disorder specific measures where available to evaluate clinical outcomes. This may therefore lead to tailored prediction of clinical outcome for individual disorders.

Most importantly, this study illustrates the power of IECBT as a data collection and research platform. One advantage of the in-service data collection method used here is that replication of these findings is possible in a way that is often cost-prohibitive in clinical trials. Analysis of subsequent cohorts may be used to increase the amount of data available for performing regression analysis, and may also add to the scientific knowledge of CBT's change mechanisms by using natural language processing to analyze therapy session transcripts collected via IECBT's unique method. Analysis of therapy sessions transcripts will allow researchers to identify which CBT change mechanisms are associated with good clinical outcomes, but also encourage good clinical practice by testing therapists' adherence to CBT protocols.

Understanding predictors of good clinical outcomes will facilitate development of patient focused, stratified/stepped-care allocation models and enable the development of enhanced therapeutic protocols. Data derived from an outcomes measurement framework is also of potential value to providers, who can adapt their services to better meet the needs of their patients and consistently monitor service quality. In England, widespread use of IECBT, coupled with continuous monitoring of clinical effectiveness using an outcomes measurement framework, has enabled systematic improvements in the quality and consistency of care delivery and enabled a transition from fee-for-service to fee-for-value payment models. Adopting a similar model in the US healthcare system could play a critical role in improving access to services whilst managing costs.

### Example 2

Methods. Data were analysed from patients receiving IECBT for the treatment of a mental health disorder between April 2015 and March 2016. IECBT was delivered using a commercial package, originally developed for and currently used in the English National Health Service, provided by leso Digital Health (http://uk.iesohealth.com). Patients self-referred or were referred by a primary healthcare worker directly to the service in the regions of Surrey, West Kent, Camden and East Riding of Yorkshire. Patients registered with the service using an online registration form or over the phone. The selection of the geographical areas was determined by those where the provider currently accepts self-referrals. There is nothing to suggest that patients in these geographical areas differ from those in other areas, or that the findings reported herein would not be generalizable. Patients reporting suicidal intent during registration or at any point during the episode of care were appropriately advised online by their therapist or another member of the clinical team, and signposted to specialist services accordingly. In exceptional circumstances of immediate or serious risk patients were contacted over the phone by their therapist or a clinical supervisor.

After registration, patients were assigned to a qualified CBT therapist accredited by the British Association for Behavioural & Cognitive Psychotherapies (BABCP). Initial assessments were carried out in an online therapy room via one-to-one real-time written conversation (instant synchronous messaging) after which the therapist assigned the patient a diagnosis, and NICE approved disorder specific CBT treatment protocols, based on Roth and Pilling's CBT competences framework, were delivered during weekly sessions. Treatment duration was determined by the therapist based on their clinical judgement, with typical treatment length between 6 and 8 sessions. Between treatment appointments/sessions asynchronous messages and homework tasks were exchanged between therapist and patient, promoting engagement and adherence to evidence-based treatment models. All communication between therapist and patient was carried out exclusively online through leso's proprietary platform following internationally recognised standards for information security (ISO 27001; https://www.iesohealth.com/en-gb/le gal/iso-certificates).

Clinical outcomes in the IECBT group were referenced against reported outcomes for patients referred to IAPT services in the same time period and same regions where IECBT was offered. Patients in the reference group received care as usual, comprising high- and low-intensity treatments, face-to-face and online therapy services, including IECBT (IAPT annual report for 2015/2016, publicly available at http://content.digital.nhs.uk/iaptreports).

The information captured through IAPT's minimum dataset, including IECBT, is intended to support monitoring of implementation and effectiveness of national policy/legislation, policy development, performance analysis and benchmarking, national analysis and statistics, and national audit of IAPT services. At registration patients agree to the services' terms and conditions, including use of anonymised data for audit purposes and to support research, including academic publications or conference presentations.

Outcomes measures. Clinical outcomes were measured in terms of clinical recovery and improvement, defined following IAPT guidelines. According to IAPT convention, these measures are defined for patients undergoing a minimum of two sessions of therapy. This is the minimum dose of therapy a patient must receive such that pre- and post-treatment scores are collected and clinical change can be estimated. Patients undergoing a minimum of two sessions of therapy may otherwise be deemed ‘engaged’.

Clinical recovery and improvement were calculated based on two severity measures completed by the patient at initial assessment and for every therapy session (completion rate 95%): PHQ-9 and GAD-7, corresponding to depressive and anxiety symptoms respectively.

The PHQ-9 is a 9-item measure designed to facilitate screening and severity assessment of depression, with a score ranging from 0 to 27 and with a recommended cut-off of 10 or more for distinguishing patients considered to be suffering from clinically significant depressive symptoms. A reduction of 6 points or more on the PHQ-9 scale between two time points is indicative of statistically reliable improvement in symptom severity. The GAD-7 is a 7-item screening and severity measure for generalized anxiety disorder, with a score ranging from 0 to 21 and with a recommended cut-off of 8 or more for distinguishing patients considered to be suffering from clinically significant anxiety symptoms. A reduction of 4 points or more on the GAD-7 scale between two time points is indicative of statistically reliable improvement in symptom severity. If a patient scores above the clinical threshold for one or both of these measures at initial assessment (i.e. 10 or above for PHQ-9 and/or 8 or above for GAD-7), they are classed as meeting caseness at assessment. Other symptom severity measures, such as severity scores for subtypes of anxiety disorders, were not examined as only PHQ-9 and GAD-7 are mandatorily collected within the IAPT framework.

For patients undergoing two or more therapy sessions, the difference between scores at initial assessment and last treatment session for PHQ-9 and GAD-7 was used to determine patients' recovery status. Recovery is a binary measure. Under IAPT guidelines, patients with two or more therapy sessions who move from above caseness at assessment to below caseness on both the PHQ-9 and GAD-7 scales at the last treatment session are classed as recovered. The recovery rate for a group of patients is calculated as number of patients recovered, divided by number of patients at caseness at initial assessment.

Improvement is also a binary measure. Under IAPT guidance, patients with two or more therapy sessions who show a significant reduction in at least one of the outcome measures from assessment to the last treatment session, whilst not showing a significant increase in the other outcome measure, were classed as improved (i.e. a decrease of 6 points or more in the PHQ-9 and/or 4 points or more in the GAD-7, whilst not simultaneously showing an increase of 6 points or more in the PHQ-9 and/or 4 points or more in the GAD-7). The improvement rate for a group of patients is calculated as number of patients improved, divided by number of patients with two or more therapy sessions. Patients who simultaneously improve and recover are classed as reliably recovered.

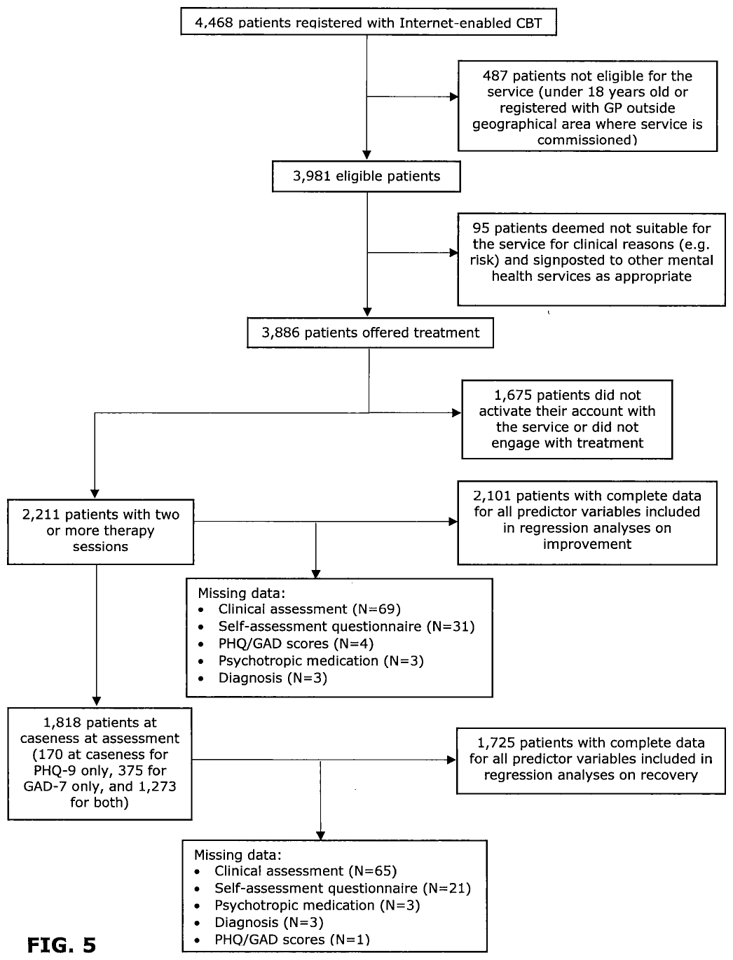

Sample size. A total of 4,468 patients registered with the IECBT service between April 2015 and March 2016. Of these, 487 patients (11%) did not meet the eligibility criteria (over 18 years old, registered with a GP in the geographical region where the service is commissioned) and were signposted to other mental health services as appropriate. Of the remaining 3,981 eligible patients, 95 (2%) were deemed not suitable for the service for clinical reasons (e.g. risk, axis-II disorder) and were signposted to other mental health services as appropriate. A total of 3,886 patients were offered treatment, of which 2,211 (57%) had two or more treatment sessions. These 2,211 patients may be termed ‘engaged’. Out of these 2,211 patients, 1,818 (82%) were at caseness at assessment (170 at caseness according to PHQ-9 only, 375 at caseness for GAD-7 only, and 1,273 at caseness for both; FIG. 5). A comparison of demographics between patients undergoing two or more therapy sessions and patients who drop-out before this point can be found in Table 4.

Between April 2015 and March 2016 a total of 45,560 referrals were received by IAPT services in the same regions where IECBT was offered. 19,325 patients were discharged in this time period having had two therapy sessions or more, of which 17,470 (90%) were at caseness at assessment.

Data analyses focusing on the improvement metric were conducted on data from patients with two or more therapy sessions, whilst analyses focusing on the recovery metric were conducted on data from patients at caseness at assessment who also had two or more therapy sessions.

Regression analyses—predictors of clinical response in IECBT. Logistic regression analyses were performed in R to identify significant predictors of recovery and improvement in patients receiving IECBT, based on patient demographics and service variables. Demographic variables included patient gender, age, severity, diagnosis, whether or not the patient suffered from a long-term physical condition, and whether or not the patient was taking psychotropic medication (e.g.: anti-depressants or anxiolytics) at the start of treatment. Service variables comprised data pertaining to a patient's course of treatment, including waiting times between various stages in the patient journey, treatment duration and number of scheduled appointments the patient failed to attend.

Given the nature of the statistical models employed, record sets were included only for cases with complete data for all predictor variables. Of the initial sample of 2,211 patients with two therapy sessions and 1,818 patients at caseness at assessment, 95% had complete data for all predictor variables and were included in the analyses (N=2,101 for improvement analysis, N=1,725 for recovery analysis; FIG. 5).

Continuous predictor variables were scaled and centred to the mean. Multicollinearity analyses were performed to investigate potential correlations between predictor variables. Statistical significance was defined as P<0.05 two-tailed, uncorrected.

Comparative analysis of clinical outcomes. Although inferential analysis of comparative clinical effectiveness is not possible in the present study due to the lack of a face-to-face control group, publicly available IAPT data makes it possible to reference IECBT clinical outcomes against averages for the same time period and geographical regions. Patients with two or more therapy sessions in the IECBT group (N=2,211) were matched to the IAPT reference group (N=19,325) for severity (PHQ-9 and GAD-7 scores at assessment), using a multivariate rejection sampling algorithm implemented in R. Lack of publicly available distribution data for other variables means it was not possible to match the two groups for other potentially relevant variables such as age, diagnosis and presence of long-term physical comorbidities. Clinical outcomes of the severity matched IECBT group relative to IAPT are reported herein.

Results. Regression analyses—predictors of clinical response in IECBT. Logistic regression analyses identified the presence of long-term physical conditions, initial GAD-7 scores, waiting time for assessment, total number of treatment sessions and patient age as significant predictors of improvement (Table 5). Apart from waiting time for assessment, these variables were also identified as significant predictors of recovery, in addition to initial PHQ-9 scores (Table 6).

Results show that patients with long-term physical conditions are less likely to show good clinical outcomes compared to patients without long-term conditions (Tables 5 and 6). Patients with higher severity scores at assessment are also less likely to show clinical recovery (Table 6). However, in line with what can be observed from Table 5, results suggest that patients with higher GAD-7 scores at assessment have higher likelihood of showing clinical improvement.

A significant positive association between patient age and likelihood of good clinical outcomes was also observed. This association was explored further in a post-hoc analysis which revealed a significant negative correlation between patient age and severity (Age and PHQ-9: r=−0.09, t=−4.02, df=2102, P<0.001; Age and GAD-7: r=−0.13, t=−5.98, df=2102, P<0.001), as well as a weak but significant positive correlation between patient age and number of treatment sessions (r=0.05, t=2.10, df=2102, P=0.036).

Finally, results show that patients who have undergone a larger number of therapy sessions are more likely to show good clinical outcomes (Tables 5 and 6). However, post-hoc analyses showed no significant association between treatment duration and clinical outcomes in patients with 5 or more sessions. Clinical outcomes rates were optimal and less variable for treatment durations of 5 to 9 sessions (51% of patients with more than two sessions, recovery rate: 57%-60%; improvement rate: 67%-72%). Clinical outcomes for patients with more than 2 but fewer than 5 treatment sessions were significantly lower and more variable (14% of all patients with more than two sessions, recovery rate: 27%-53%; improvement rate: 42%-61%).

Tests of the full models against constant-only models were significant for both regression analyses (Improvement regression model: X2(19)=195.95, P<0.001; Recovery regression model: X2(19)=278.36, P<0.001). Additionally, multicollinearity analyses revealed variance inflation factors smaller than 2 for all predictor variables. This is the standard threshold value for indicating the presence of multicollinearity in this type of analysis, thus confirming that regression models were not affected by the presence of multicollinearity.

IAPT's improvement and recovery metrics are, by definition, biased by initial symptom severity, which confounds interpretation of the results. In a post-hoc regression analysis investigating predictors of clinical outcomes whilst controlling for artefactual relations with initial severity, percent improvement was defined as a 25% decrease in scores for both the PHQ-9 and the GAD-7. Similar to IAPT convention, a patient was classed as achieving percent improvement if they showed a 25% decrease in scores in one or both scales, without showing deterioration in either scale. Results of this analysis show broadly similar results to the analysis on predictors of improvement as defined according to IAPT convention, but they no longer show the significant association between initial severity scores and percent improvement (Table 7).

Following IAPT convention, improvement, recovery and percent improvement metrics were defined combining the PHQ-9 and GAD-7 scales. While this allows for a more comprehensive characterisation of the patients, who often present with a combination of depressive and anxiety features, these are two separate scales, measuring different constructs. It can be hypothesised that patient and service variables may impact the likelihood of good clinical outcomes for each scale differently. Post-hoc regression analyses investigating predictors of percent improvement for each scale separately are presented in Tables 8 and 9.

Clinical outcomes were adjusted for symptom severity and benchmarked against national audit comparator data. Variations were observed in the likelihood of improvement and recovery with PHQ-9 and GAD-7 scores at assessment, for patients treated with IECBT. Regression analyses results showed that more severe patients, with higher PHQ-9 and GAD-7 scores at assessment, have lower likelihoods of recovery but equivalent or higher likelihood for improvement. Accordingly, IECBT clinical improvement and recovery data were benchmarked against severity-matched cohorts. Severity-matched patients treated with IECBT showed similar improvement and recovery rates relative to IAPT patients, as well as a similar magnitude of symptom reduction, pre- and post-treatment (Table 10). Although classical significance testing was avoided due to bias in favour of rejecting the null hypothesis for large sample sizes, effect sizes and 95% confidence intervals are presented (Table 10). Despite some isolated differences in disorder distribution across the two cohorts, the observed odds ratios and effect sizes were generally small for most variables (27). Together with differences in clinical outcomes of less than 1% and differences in magnitude of symptom reduction of 0.6 points or less between the two groups, these results suggest that differences between the two groups in clinical outcomes and score reduction are unlikely to be meaningful.

Discussion. This is the first real-world (non-RCT (randomized controlled trial)) report of clinical outcomes data for patients with depression and anxiety treated using internet-enabled CBT. The first evidence of clinical efficacy of IECBT in depression was published in the Lancet in 2009 (Kessler D, Lewis G, Kaur S, Wiles N, King M, Weich S, et al. Therapist-delivered internet psychotherapy for depression in primary care: a randomised controlled trial. Lancet. 2009; 374(9690):628-34). These extended data, including both depression and anxiety disorders, offer an example of translational research put into practice and successfully deployed at scale. The application of the resultant dataset in advancing understanding of clinical and demographic variables associated with response to treatment suggests that there is value in data enabled mental health services as platforms for clinical research. Knowledge acquired with these tools can be used to refine service specifications and develop personalized treatment programmes, as part of a quality improvement cycle aiming to drive up standards in mental healthcare.

Main findings. Regression analyses revealed a significant association between initial psychometric scores and likelihood of recovery, with higher scores associated with lower recovery rates. By definition, patients recover by going below the caseness threshold for both PHQ-9 and GAD-7. Therefore it is not unexpected that patients whose initial scores are closer to that threshold have higher chances of recovery. This does however raise the question of whether recovery alone is a suitable index to measure clinical outcome, as the recovery metric does not take into account whether the observed reduction in severity is greater than the measurement error of the scales. Conversely the improvement index is a measure of whether or not a reduction in severity is statistically reliable, regardless of caseness, and may therefore be a better metric for widespread use. In the present study, patients with higher initial scores are more likely to show clinical improvement, as validated by the results of the regression analysis, where patients with higher initial GAD-7 scores show higher likelihoods of improvement (Table 5). IAPT's reliable recovery index is a composite metric measuring whether a patient recovered whilst simultaneously showing a statistically reliable reduction in severity. Although by definition this metric may be less susceptible to bias in favour of patients who are near the recovery threshold, it will still be biased against patients with higher severity scores at assessment, who will be less likely to cross the recovery threshold. To investigate predictors of clinical outcomes whilst controlling for artefactual relations with initial severity, we conducted a post-hoc analysis investigating predictors of percent improvement. Results show broadly similar results to the regression analysis on predictors of improvement as defined according to IAPT convention, but the significant association with initial severity scores is no longer present with the percent improvement measure (Table 7). Whilst differences in recovery rate with severity may be expected in this context, they may also indicate the presence of nonspecific treatment effects. Future strategies to improve treatment effectiveness should therefore be aimed at boosting recovery of more severe patients, including increased session frequency at the start of treatment, or the use of specific CBT protocols for severe depression.

Regression analyses on improvement and recovery also revealed significant associations between clinical outcomes and age, presence of a long-term physical condition and number of therapy sessions. Results show that greater age is associated with better clinical outcomes, in contrast with previous research showing lower effectiveness of CBT in older adults. However, it is important to note that in the present study the mean age of the patient cohort was 36 years, whilst previous research on the effects of CBT on older adults focused on adults over the age of 55 (Gould R L, Coulson M C, Howard R J. Efficacy of cognitive behavioral therapy for anxiety disorders in older people: A meta-analysis and meta-regression of randomized controlled trials. Vol. 60, Journal of the American Geriatrics Society. 2012. p. 218-29). Older adults are more likely to be affected by age-related cognitive decline and physical comorbidities that may directly influence CBT outcomes but are not prevalent factors in the current cohort. Post-hoc analyses on predictors of percent improvement reveal age to be a positive predictor of likelihood of percent improvement, similar to what was observed for the analysis on improvement defined under IAPT's convention. This suggests that despite a significant negative correlation between patient age and severity, the association between age and clinical outcomes is not driven by differences in severity across the age range in this particular cohort. A weak but significant positive correlation between patient age and number of treatment sessions, as well as higher mean age of patients with two or more therapy sessions (Table 4), suggests that in this particular cohort older patients may be less likely to drop-out at earliest stages of treatment therefore benefitting from a larger therapeutic dose, and consequently be more likely to achieve positive clinical outcomes.

In this cohort it was also observed that patients with long-term physical conditions were less likely to show good clinical outcomes. This finding is unsurprising given that long-term physical conditions are often associated with comorbid mental health problems and complex psychological issues, which may themselves be chronic in nature and often treatment resistant. Lower probability of response to treatment may signal the need for tailored, condition-specific CBT models, so patients can be helped to deal with mental and physical symptoms in an integrated fashion. Alternatively, disease-specific measures may be utilized, which may reflect the complexities of a particular physical disease and provide differential indicators of clinical improvement in these cases.

Service variables shown to be associated with the likelihood of good clinical outcomes included higher number of therapy sessions and reduced waiting time for assessment. Although these findings are supported by similar reports in the literature (Clark D M, Canvin L, Green J, Layard R, Pilling S, Janecka M. Transparency about the outcomes of mental health services (IAPT approach): an analysis of public data. Lancet. 2018; 391(10121), 679-686), care should be taken when drawing causal inferences. At first glance these results convey the impression that longer courses of treatment are associated with better clinical outcomes. However, an alternative explanation is that patients who do not adhere to their treatment plan and drop-out at an earlier stage during the course of treatment, therefore receiving a sub-therapeutic dose, are less likely to achieve good clinical outcomes. This hypothesis is supported by results of post-hoc analyses showing that clinical outcome rates were optimal and less variable for treatment durations of 5 to 9 sessions, whilst for patients with more than 2 but fewer than 5 treatment sessions significantly lower and more variable clinical outcomes were observed. Difficulties with engagement leading to poor clinical outcomes may be particularly relevant in patients with more severe depressive symptoms, who by the nature of their condition may lack motivation to attend treatment sessions and generally adhere to their treatment plan.

Limitations. A numerical comparison of IECBT clinical outcomes against IAPT's averages in the present study suggests that IECBT is as effective as standard care. The comparison between these two groups is presented to demonstrate general equivalence of IECBT and IAPT services, building on previous results from a clinical trial of IECBT (Kessler et al., 2009) and supporting the effectiveness of this therapy modality in a real-world clinical setting. However, it is important to note there are several limitations for this analysis and caution should be taken not to over-interpret these findings. First, since this was an audit study and not a randomized controlled trial, group comparisons between patients receiving IECBT and IAPT patients are open to the effects of selection bias. Second, although the IECBT group was matched to the reference group for severity, the aggregated nature of the data published in IAPT's annual reports means that it was not possible to use propensity analyses or selection algorithms to better match the patients who got IECBT to that subset of the patients in IAPT who were most similar to them. Third, whilst all patients in the IECBT group received CBT, patients in the IAPT reference group received a range of different therapy types, including IECBT. IECBT is not suitable for all patients, including those at risk and those who are not literate, not fluent English speakers or who do not have access to an internet-connected device. It can be hypothesized that differences in therapy type, together with potential cohort differences in other uncontrolled variables such as presence of secondary comorbid mental health conditions, IQ and socio-economic status, may also account for variance in clinical outcomes.

A positive aspect of the in-service data collection method used here and in other IAPT services is that replication of these findings is possible in a way that is often cost-prohibitive in clinical trials. The findings, methods and systems disclosed herein are expected to be generalizable to other cohorts, and also add to the scientific knowledge of effective CBT change mechanisms. Understanding predictors of good clinical outcomes is expected to facilitate development of improved, patient-focused, stratified/stepped-care allocation models and also enable the development of enhanced therapeutic protocols. Data derived from an outcomes-measurement framework is also of potential value to providers (including services, employers and individual therapists), who can adapt their services to better meet the needs of their patients and consistently monitor service quality and encourage accountability.

In England, continuous monitoring of clinical effectiveness using an outcomes measurement framework, has enabled systematic improvements in the quality and consistency of care delivery and enabled a transition from fee-for-service to fee-for-value payment models. In the US, as the Centres for Medicare and Medicaid Services begin to implement value-based payment models, the importance of understanding what and why treatments work, and what their clinical and economic impact is, becomes evident. Translating the IAPT model, including digital approaches, not only into the US but worldwide could have a dual advantage, improving the quality and accountability of mental healthcare whilst reducing cost by enabling a shift towards capitated and fee-for-value payment models.

IECBT is classed as a high-intensity therapy and can be used to treat more severe patients, relative to other self-guided and guided self-help online CBT modalities which are classed as low-intensity interventions and therefore only suitable for patients with milder presentations. Previous research investigating predictors of clinical outcomes for low-intensity guided self-help interventions has shown that higher levels of adherence to treatment and treatment credibility are associated with higher rates of improvement and lower post-treatment scores. This highlights the importance of investigating predictors of clinical outcomes in response to high-intensity online interventions like IECBT, where the synchronous, yet anonymous, nature of the interaction between therapist and patient may promote treatment credibility and patient adherence to treatment protocol.

Knowledge of which patient and service variables are associated with good clinical outcomes can be used to develop personalized treatment programmes, as part of a quality improvement cycle aiming to drive up standards in mental healthcare. This study exemplifies translational research put into practice and deployed at scale in the UK, demonstrating the value of technology-enabled treatment delivery not only in facilitating access to care, but in enabling accelerated data capture for clinical research purposes.

### Example 3

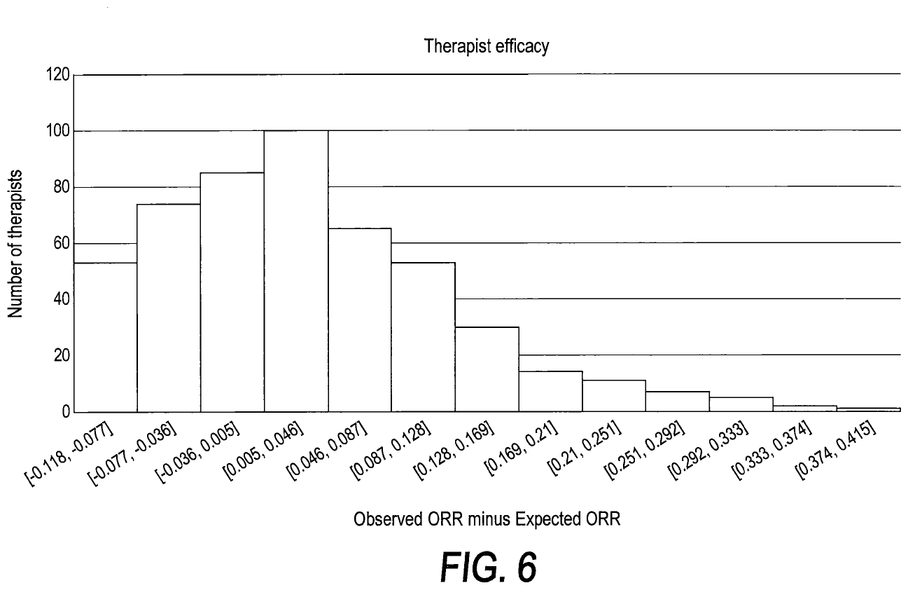

Preferential allocation of patients to the most effective therapists. Method: Patient demographics data including but not limited to the variables: symptom severity (determined by patient self-reported responses to PHQ-9 and GAD-7 questionnaires according to standard procedures), presence of a long-term physical condition (physical comorbidity), age, gender and source of referral, were collected for patients referred to 500 therapists providing internet-enabled cognitive behavioural therapy. A regression model was constructed to predict patients' likelihood of response to treatment based on these variables. The patients were then grouped by each of the therapists administering the treatment. ‘Predicted recovery rate’ for each therapist was calculated as the mean prediction of psychological therapy outcome for the patients treated by that therapist (denoted ‘expected Overall Recovery Rate’ (expected ORR)), based on patient demographic variables only, as above. The actual recovery rate achieved by each therapist at the end of treatment was calculated as the mean observed psychological therapy outcome for the patients treated by that therapist (denoted ‘observed Overall Recovery Rate’ (observed ORR)). The difference between actual (observed) and predicted psychotherapy outcome attributed to each therapist was calculated (observed ORR minus expected ORR), and therapists were ranked according to these differences. The most effective therapists were those who achieved actual recovery rates higher than predicted, and the least effective therapists were those who achieved actual recovery rates lower than predicted. Exemplary data for 21 anonymized therapists are presented in Table 11 below; FIG. 6 is a histogram illustrating the data for all 500 therapists.

The data in Table 11 is ranked by difference between the observed ORR and expected ORR, such that the most effective therapists are towards the top of the table (Observed ORR minus Expected ORR′ having a positive value), and the least effective therapists are towards the bottom of the table (Observed ORR minus Expected ORR′ having a negative value). The data for one therapist in Table 11 (Anonymized Therapist ID 3117530F) indicated that that therapist performed exactly according to expectation based on the regression model and data inputted.

This information is then used by the method to preferentially allocate patients, or just the most severely affected patients, to the most effective therapists. The therapists ranked as ‘least effective’ by the method can be allocated to a therapist support protocol e.g. the provision of additional support materials, retraining, additional supervision etc. Thereby the quality (effectiveness) of the therapy offered to patients increases over time when using the methods disclosed herein.

### Example 4

Quality control of new therapists. Method: Using a range of patient demographics including but not limited to symptom severity, presence of a physical comorbidity (long-term physical condition), age, gender and source of referral, a regression model is constructed to predict patients' likelihood of response to treatment based on these variables. For each new therapist, a prediction of psychological therapy outcome is obtained for one or more patient according to the methods described above. Where there is more than one patient, this may be expressed as an expected overall recovery rate (ORR). Subsequent to treatment of the one or more patient by the new therapist, an observation of the actual psychological therapy outcome is obtained for each of the same one or more patient(s). Again, where there is more than one patient this may be expressed as an observed ORR. The difference between actual and predicted recovery rate is calculated for each therapist (e.g. observed ORR minus expected ORR). Where the observed value minus the predicted value is positive (e.g. observed ORR is greater than expected ORR), the therapist is deemed to be ‘most effective’, whereas where the observed value minus the predicted value is negative (e.g. observed ORR is less than expected ORR), the therapist is deemed to be ‘least effective’.

This information is then used by the method, for example to preferentially allocate patients to the most effective new therapists, and/or to offer additional support to the least effective new therapists e.g. additional support materials, retraining by a more experienced therapist, additional supervision etc.

### Example 5

A new patient presents to the IECBT provider. Medical history and demographic data (e.g. for variables gender, age, whether or not the patient has a comorbid long-term physical condition, employment status, whether or not the patient is taking psychotropic medication, whether or not a patient is currently pregnant, or has been pregnant or given birth in the previous 12 months) for the patient are obtained via a questionnaire delivered via the patient's computer interface e.g. a secure mobile phone or tablet application. The patient is also asked to complete the GAD-7 and PHQ-9 questionnaires. All the data collected from the patient is sent to a cloud-based server, where a regression model that uses the characteristics of patients who have already been treated and their known treatment outcomes is used in order to assign a score to each data variable available for the new patient. These scores are combined to calculate an aggregate score for the patient. The aggregate score is compared with a scale to determine a prediction of psychological therapy outcome for the patient. The prediction of psychological therapy outcome is then used to determine a suitable treatment protocol for that patient, the determined treatment protocol being outputted to a computer interface accessible to one or more of a therapist, a therapy supervisor, a therapy service and/or the payor for the therapy (i.e. insurance provider). The determined treatment protocol is then initiated on the patient's interface, e.g. the secure mobile phone or tablet app.

Therefore, the present invention is well adapted to attain the ends and advantages mentioned as well as those that are inherent therein. The particular embodiments disclosed above are illustrative only, as the present invention may be modified and practiced in different but equivalent manners apparent to those skilled in the art having the benefit of the teachings herein. Furthermore, no limitations are intended to the details of construction or design herein shown, other than as described in the claims below. It is therefore evident that the particular illustrative embodiments disclosed above may be altered, combined, or modified and all such variations are considered within the scope and spirit of the present invention. The invention illustratively disclosed herein suitably may be practiced in the absence of any element that is not specifically disclosed herein and/or any optional element disclosed herein. While compositions and methods are described in terms of “comprising,” “containing,” or “including” various components or steps, the compositions and methods can also “consist essentially of or” consist of the various components and steps. All numbers and ranges disclosed above may vary by some amount. Whenever a numerical range with a lower limit and an upper limit is disclosed, any number and any included range falling within the range is specifically disclosed. In particular, every range of values (of the form, “from about a to about b,” or, equivalently, “from approximately a to b,” or, equivalently, “from approximately a-b”) disclosed herein is to be understood to set forth every number and range encompassed within the broader range of values. Also, the terms in the claims have their plain, ordinary meaning unless otherwise explicitly and clearly defined by the patentee. Moreover, the indefinite articles “a” or “an,” as used in the claims, are defined herein to mean one or more than one of the element that it introduces. Various further aspects and embodiments of the present invention will be apparent to those skilled in the art in view of the present disclosure. All documents mentioned in this specification are incorporated herein by reference in their entirety. “and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein. Unless context dictates otherwise, the descriptions and definitions of the features set out above are not limited to any particular aspect or embodiment of the invention and apply equally to all aspects and embodiments which are described.

### Tables

TABLE 4
Demographics comparison between patients undergoing two
or more treatment sessions and patients who drop-out before
this stage. StartPhq9 - PHQ-9 score at assessment; StartGad7 -
GAD-7 score at assessment.

| | Less than 2 | 2 or more |
|---|---|---|
| | treatment | treatment |
| | sessions | sessions |
| | (N = 1,675) | (N = 2,211) |
| Gender | | |
| Male | 30% | 27% |
| Female | 69% | 72% |
| Unknown/Not stated | 1% | 1% |
| Age | 35.2 years | 36.8 years |
| Long Term Physical Conditions | | |
| No | 20% | 34% |
| Yes | 15% | 21% |
| Unknown/Not stated | 64% | 45% |
| StartPhq9 | 13.5 | 12.7 |
| StartGad7 | 12.1 | 11.9 |

TABLE 5
Results of logistic regression analysis investigating predictors
of improvement in the Internet-enabled CBT cohort. Gender “Male”,
Long Term Physical Conditions “No”, Diagnosis “Anxiety”
and Psychotropic Medication “Prescribed Not Taking” were reference
classes for the categorical variables. Diagnosis = “Depression”
encompasses patients diagnosed with depressive episode, dysthymia or
recurrent depressive disorder. Diagnosis = “Anxiety” encompasses
patients diagnosed with agoraphobia, generalised anxiety disorder,
hypochondriacal disorder, obsessive-compulsive disorder, panic disorder,
post-traumatic stress disorder, social phobia, specific phobia or anxiety
disorder unspecified. Diagnosis = “Other” encompasses all
diagnoses not otherwise listed (e.g.: chronic pain, bereavement, eating
disorders). StartPhq9 - PHQ-9 score at assessment; StartGad7 - GAD-7
score at assessment; Waiting SAQ - time between referral and patient
completing self-assessment questionnaire; WaitingAssignment - waiting
time from patient completing the self-assessment questionnaire and
therapist assignment; WaitingContact - waiting time between therapist
assignment and first contact by the therapist; WaitingAssessment -
waiting time between first contact from the therapist and clinical
assessment appointment; WaitingTreatment - waiting time between
clinical assessment and first therapy session; NumberSessions -
total number of therapy sessions attended by the patient; NumberDNA -
number of scheduled appointments the patient failed to attend.
Predictors of Improvement in Internet-enabled CBT (N = 2,101)

| | | | | | | | |
|---|---|---|---|---|---|---|---|
| | | | | Wald's | | | |
| | | | | statistic | | N | Improvement |
| Predictor variable | Mean/Prevalence | b | SE | (z²) | P-value | (subgroup) | rate |

Gender

| Male | 27.3% | | — | — | — | — | 574 | 60.1% |
|---|---|---|---|---|---|---|---|---|
| Female | 72.1% | | 0.04 | 0.11 | 0.15 | .698 | 1,514 | 62.2% |
| Unknown/Not stated | 0.6% | | −0.24 | 0.59 | 0.17 | .682 | 13 | 53.8% |
| Age | 36.8 | years | 0.12 | 0.05 | 5.87 | .015 * | — | — |

Long Term Physical Conditions

| | | | | | | | |
|---|---|---|---|---|---|---|---|
| No | 34.3% | — | — | — | — | 721 | 60.9% |
| Yes | 20.8% | −0.32 | 0.14 | 5.29 | .021 * | 436 | 59.9% |
| Unknown/Not stated | 44.9% | 0.02 | 0.11 | 0.05 | .819 | 944 | 62.9% |

Diagnosis

| | | | | | | | |
|---|---|---|---|---|---|---|---|
| Anxiety | 42.1% | — | — | — | — | 885 | 61.4% |
| Depression | 22.6% | 0.03 | 0.13 | 0.04 | .851 | 474 | 60.1% |
| Other | 35.3% | 0.13 | 0.11 | 1.30 | .254 | 742 | 62.8% |

Psychotropic Medication

| Prescribed Not Taking | 8.1% | | — | — | — | — | 171 | 59.6% |
|---|---|---|---|---|---|---|---|---|
| Prescribed Taking | 38.3% | | 0.24 | 0.18 | 1.78 | .182 | 805 | 63.6% |
| Not Prescribed | 52.5% | | 0.26 | 0.18 | 2.05 | .152 | 1,103 | 60.4% |
| Unknown/Not stated | 1.0% | | 0.23 | 0.49 | 0.21 | .649 | 22 | 63.6% |
| StartPhq9 | 12.7 | | 0.05 | 0.07 | 0.50 | .479 | — | — |
| StartGad7 | 11.9 | | 0.53 | 0.06 | 70.91 | <.001 *** | — | — |
| WaitingSAQ | 3.0 | days | 0.01 | 0.06 | 0.02 | .894 | — | — |
| WaitingAssignment | 0.8 | days | −0.04 | 0.06 | 0.44 | .508 | — | — |
| WaitingContact | 1.1 | days | −0.07 | 0.08 | 0.93 | .336 | — | — |
| WaitingAssessment | 7.6 | days | −0.14 | 0.05 | 7.54 | .006 ** | — | — |
| WaitingTreatment | 8.9 | days | −0.02 | 0.05 | 0.18 | .671 | — | — |
| NumberSessions | 5.5 | sessions | 0.29 | 0.05 | 32.09 | <.001 *** | — | — |
| NumberDNA | 0.5 | sessions | −0.01 | 0.05 | 0.06 | .813 | — | — |

TABLE 6
Results of logistic regression analysis investigating predictors
of recovery in the Internet-enabled CBT cohort. Gender “Male”,
Long Term Physical Conditions “No”, Diagnosis “Anxiety”
and Psychotropic Medication “Prescribed Not Taking” were
reference classes for the categorical variables. Diagnosis =
“Depression” encompasses patients diagnosed with depressive
episode, dysthymia or recurrent depressive disorder. Diagnosis =
“Anxiety” encompasses patients diagnosed with agoraphobia,
generalised anxiety disorder, hypochondriacal disorder, obsessive-compulsive
disorder, panic disorder, post-traumatic stress disorder, social
phobia, specific phobia or anxiety disorder unspecified. Diagnosis =
“Other” encompasses all diagnoses not otherwise listed (e.g.:
chronic pain, bereavement, eating disorders). StartPhq9 - PHQ-9
score at assessment; StartGad7 - GAD-7 score at assessment; Waiting
SAQ - time between referral and patient completing self-assessment
questionnaire; WaitingAssignment - waiting time from patient completing
the self-assessment questionnaire and therapist assignment;
WaitingContact - waiting time between therapist assignment and first
contact by the therapist; WaitingAssessment - waiting time between
first contact from the therapist and clinical assessment appointment;
WaitingTreatment - waiting time between clinical assessment and first
therapy session; NumberSessions - total number of therapy sessions
attended by the patient; NumberDNA - number of scheduled appointments
the patient failed to attend.
Predictors of Recovery in Internet-enabled CBT (N = 1,725)

| | | | | | | | |
|---|---|---|---|---|---|---|---|
| | | | | Wald's | | | |
| | | | | statistic | | N | Recovery |
| Predictor variable | Mean/Prevalence | b | SE | (z²) | P-value | (subgroup) | rate |

Gender

| Male | 26.5% | | — | — | — | — | 457 | 51.0% |
|---|---|---|---|---|---|---|---|---|
| Female | 73.0% | | 0.15 | 0.12 | 1.62 | .202 | 1,259 | 50.8% |
| Unknown/Not stated | 0.5% | | −0.14 | 0.70 | 0.04 | .846 | 9 | 44.4% |
| Age | 36.3 | years | 0.23 | 0.06 | 18.05 | <.001 *** | — | — |

Long Term Physical Conditions

| | | | | | | | |
|---|---|---|---|---|---|---|---|
| No | 33.2% | — | — | — | — | 573 | 55.8% |
| Yes | 22.0% | −0.37 | 0.15 | 5.88 | .015 * | 379 | 43.5% |
| Unknown/Not stated | 44.8% | −0.14 | 0.12 | 1.32 | .251 | 773 | 50.6% |

Diagnosis

| | | | | | | | |
|---|---|---|---|---|---|---|---|
| Anxiety | 40.8% | — | — | — | — | 703 | 53.3% |
| Depression | 23.6% | −0.04 | 0.15 | 0.07 | .796 | 407 | 45.9% |
| Other | 35.7% | 0.02 | 0.13 | 0.02 | .875 | 615 | 51.1% |

Psychotropic Medication

| Prescribed Not Taking | 8.8% | | — | — | — | — | 151 | 47.0% |
|---|---|---|---|---|---|---|---|---|
| Prescribed Taking | 41.7% | | 0.01 | 0.20 | 0.01 | .942 | 719 | 46.3% |
| Not Prescribed | 48.3% | | 0.06 | 0.20 | 0.09 | .769 | 834 | 55.6% |
| Unknown/Not stated | 1.2% | | −0.49 | 0.53 | 0.84 | .360 | 21 | 38.1% |
| StartPhq9 | 14.3 | | −0.56 | 0.07 | 70.32 | <.001 *** | — | — |
| StartGad7 | 13.4 | | −0.30 | 0.06 | 24.76 | <.001 *** | — | — |
| WaitingSAQ | 3.0 | days | 0.01 | 0.06 | 0.02 | .900 | — | — |
| WaitingAssignment | 0.8 | days | 0.04 | 0.06 | 0.36 | .550 | — | — |
| WaitingContact | 1.2 | days | −0.11 | 0.07 | 2.16 | .142 | — | — |
| WaitingAssessment | 7.4 | days | −0.08 | 0.06 | 2.16 | .141 | — | — |
| WaitingTreatment | 8.8 | days | −0.02 | 0.05 | 0.18 | .674 | — | — |
| NumberSessions | 5.6 | sessions | 0.32 | 0.05 | 33.83 | <.001 *** | — | — |
| NumberDNA | 0.5 | sessions | −0.11 | 0.05 | 3.81 | .051 | — | — |

TABLE 7
Results of logistic regression analysis investigating predictors
of percent improvement in the Internet-enabled CBT cohort. Gender
“Male”, Long Term Conditions “No”, Condition “Anxiety”
and Psychotropic Medication “Prescribed Not Taking” were
reference classes for the categorical variables. Condition =
“Depression” encompasses patients diagnosed with depressive
episode, dysthymia or recurrent depressive disorder. Condition =
“Anxiety” encompasses patients diagnosed with agoraphobia,
generalised anxiety disorder, hypochondriacal disorder, obsessive-
compulsive disorder, panic disorder, post-traumatic stress disorder,
social phobia, specific phobia or anxiety disorder unspecified.
Condition = “Other” encompasses all diagnoses not otherwise
listed (e.g.: chronic pain, bereavement, eating disorders). StartPhq9 -
PHQ-9 score at assessment; StartGad7 - GAD-7 score at assessment;
Waiting SAQ - time between referral and patient completing self-
assessment questionnaire; WaitingAssignment - waiting time from
patient completing the self-assessment questionnaire and therapist
assignment; WaitingContact - waiting time between therapist assignment
and first contact by the therapist; WaitingAssessment - waiting time
between first contact from the therapist and clinical assessment
appointment; WaitingTreatment - waiting time between clinical
assessment and first therapy session; NumberSessions - total number
of therapy sessions attended by the patient; NumberDNA - number of
scheduled appointments the patient failed to attend.
Predictors of percent improvement in internet-enabled CBT (N = 2,101)

| | | | | Wald's | |
|---|---|---|---|---|---|
| | | | | statistic | |
| Predictor variable | Mean/Prevalence | B | Se | (z²) | P-value |

Gender

| Male | 27.3% | | — | — | — | — |
|---|---|---|---|---|---|---|
| Female | 72.1% | | 0.002 | 0.11 | 0.0002 | .988 |
| Unknown/not stated | 0.6% | | 0.70 | 0.78 | 0.80 | .372 |
| Age | 36.8 | years | 0.12 | 0.05 | 5.51 | .019 * |

Long term conditions

| | | | | | |
|---|---|---|---|---|---|
| No | 34.3% | — | — | — | — |
| Yes | 20.8% | −0.22 | 0.14 | 2.33 | .127 |
| Unknown/not stated | 44.9% | −0.02 | 0.11 | 0.03 | .855 |

Condition

| | | | | | |
|---|---|---|---|---|---|
| Anxiety | 42.1% | — | — | — | — |
| Depression | 22.6% | 0.04 | 0.14 | 0.10 | .753 |
| Other | 35.3% | 0.20 | 0.12 | 2.78 | .096 |

Psychotropic medication

| Prescribed not taking | 8.1% | | — | — | — | — |
|---|---|---|---|---|---|---|
| Prescribed taking | 38.3% | | 0.17 | 0.19 | 0.82 | .365 |
| Not prescribed | 52.5% | | 0.19 | 0.18 | 1.10 | .293 |
| Unknown/not stated | 1.0% | | 0.07 | 0.50 | 0.02 | .891 |
| StartPhq9 | 12.7 | | −0.13 | 0.07 | 3.49 | .062 |
| StartGad7 | 11.9 | | 0.10 | 0.07 | 2.36 | .125 |
| WaitingSAQ | 3.0 | days | −0.02 | 0.06 | 0.09 | .771 |
| WaitingAssignment | 0.8 | days | −0.05 | 0.06 | 0.78 | .378 |
| WaitingContact | 1.1 | days | −0.07 | 0.06 | 1.03 | .309 |
| WaitingAssessment | 7.6 | days | −0.14 | 0.05 | 7.86 | .005 ** |
| WaitingTreatment | 8.9 | days | −0.07 | 0.05 | 2.46 | .117 |
| NumberSessions | 5.5 | sessions | 0.26 | 0.05 | 22.78 | <.001 *** |
| NumberDNA | 0.5 | sessions | −0.03 | 0.05 | 0.41 | .521 |

TABLE 8
Results of logistic regression analysis investigating predictors of percent improvement for PHQ-9 metric in the Internet-enabled CBT cohort. Gender “Male”, Long Term Conditions “No”, Condition “Anxiety” and Psychotropic Medication “Prescribed Not Taking” were reference classes for the categorical variables. Condition = “Depression” encompasses patients diagnosed with depressive episode, dysthymia or recurrent depressive disorder. Condition = “Anxiety” encompasses patients diagnosed with agoraphobia, generalised anxiety disorder, hypochondriacal disorder, obsessive-compulsive disorder, panic disorder, post-traumatic stress disorder, social phobia, specific phobia or anxiety disorder unspecified. Condition = “Other” encompasses all diagnoses not otherwise listed (e.g.: chronic pain, bereavement, eating disorders). StartPhq9 - PHQ-9 score at assessment; Waiting SAQ - time between referral and patient completing self-assessment questionnaire; WaitingAssignment - waiting time from patient completing the self-assessment questionnaire and therapist assignment; WaitingContact - waiting time between therapist assignment and first contact by the therapist; WaitingAssessment - waiting time between first contact from the therapist and clinical assessment appointment; WaitingTreatment - waiting time between clinical assessment and first therapy session; NumberSessions - total number of therapy sessions attended by the patient; NumberDNA - number of scheduled appointments the patient failed to attend.
Predictors of Percent Improvement (PHQ-9) in Internet-enabled CBT (N = 2,101)

| | | | | | |
|---|---|---|---|---|---|
| | | | | Wald's | |
| | | | | statistic | |
| Predictor variable | Mean/Prevalence | b | SE | (z²) | P-value |

Gender

| Male | 27.3% | | — | — | — | — |
|---|---|---|---|---|---|---|
| Female | 72.1% | | −0.04 | 0.11 | 0.11 | .736 |
| Unknown/Not stated | 0.6% | | 0.98 | 0.78 | 1.58 | .210 |
| Age | 36.8 | years | 0.18 | 0.05 | 12.81 | <.001 *** |

Long Term Conditions

| | | | | | |
|---|---|---|---|---|---|
| No | 34.3% | — | — | — | — |
| Yes | 20.8% | −0.16 | 0.14 | 1.40 | .237 |
| Unknown/Not stated | 44.9% | −0.12 | 0.11 | 1.34 | .246 |

Condition

| | | | | | |
|---|---|---|---|---|---|
| Anxiety | 42.1% | — | — | — | — |
| Depression | 22.6% | 0.24 | 0.13 | 3.46 | .063 |
| Other | 35.3% | 0.35 | 0.11 | 10.06 | .002 ** |

Psychotropic Medication

| Prescribed Not Taking | 8.1% | | — | — | — | — |
|---|---|---|---|---|---|---|
| Prescribed Taking | 38.3% | | 0.32 | 0.18 | 3.29 | .070 |
| Not Prescribed | 52.5% | | 0.39 | 0.17 | 5.11 | .024 * |
| Unknown/Not stated | 1.0% | | 0.38 | 0.48 | 0.62 | .430 |
| StartPhq9 | 12.7 | | 0.06 | 0.05 | 1.13 | .287 |
| WaitingSAQ | 3.0 | days | 0.02 | 0.05 | 0.08 | .773 |
| WaitingAssignment | 0.8 | days | −0.04 | 0.05 | 0.66 | .416 |
| WaitingContact | 1.1 | days | −0.04 | 0.06 | 0.46 | .498 |
| WaitingAssessment | 7.6 | days | −0.15 | 0.05 | 9.33 | .002 ** |
| WaitingTreatment | 8.9 | days | −0.12 | 0.05 | 6.55 | .010 * |
| NumberSessions | 5.5 | sessions | 0.23 | 0.05 | 21.76 | <.001 *** |
| NumberDNA | 0.5 | sessions | −0.07 | 0.05 | 1.99 | .159 |

TABLE 9
Results of logistic regression analysis investigating predictors
of percent improvement for GAD-7 metric in the Internet-enabled CBT
cohort. Gender “Male”, Long Term Conditions “No”,
Condition “Anxiety” and Psychotropic Medication “Prescribed
Not Taking” were reference classes for the categorical variables.
Condition = “Depression” encompasses patients diagnosed
with depressive episode, dysthymia or recurrent depressive disorder.
Condition = “Anxiety” encompasses patients diagnosed with
agoraphobia, generalised anxiety disorder, hypochondriacal disorder,
obsessive-compulsive disorder, panic disorder, post-traumatic stress
disorder, social phobia, specific phobia or anxiety disorder unspecified.
Condition = “Other” encompasses all diagnoses not otherwise
listed (e.g.: chronic pain, bereavement, eating disorders). StartGad7 -
GAD-7 score at assessment; Waiting SAQ - time between referral and
patient completing self-assessment questionnaire; WaitingAssignment -
waiting time from patient completing the self-assessment questionnaire
and therapist assignment; WaitingContact - waiting time between therapist
assignment and first contact by the therapist; WaitingAssessment -
waiting time between first contact from the therapist and clinical
assessment appointment; WaitingTreatment - waiting time between clinical
assessment and first therapy session; NumberSessions - total number
of therapy sessions attended by the patient; NumberDNA - number of
scheduled appointments the patient failed to attend.
Predictors of Percent Improvement (GAD-7) in Internet-enabled CBT (N = 2,101)

| | | | | Wald's | |
|---|---|---|---|---|---|
| | | | | statistic | |
| Predictor variable | Mean/Prevalence | b | SE | (z²) | P-value |

Gender

| Male | 27.3% | | — | — | — | — |
|---|---|---|---|---|---|---|
| Female | 72.1% | | 0.08 | 0.10 | 0.59 | .441 |
| Unknown/Not stated | 0.6% | | 1.15 | 0.78 | 2.17 | .141 |
| Age | 36.8 | years | 0.08 | 0.05 | 2.79 | .095 |

Long Term Conditions

| | | | | | |
|---|---|---|---|---|---|
| No | 34.3% | — | — | — | — |
| Yes | 20.8% | −0.29 | 0.13 | 4.80 | .028 * |
| Unknown/Not stated | 44.9% | −0.05 | 0.11 | 0.25 | .614 |

Condition

| | | | | | |
|---|---|---|---|---|---|
| Anxiety | 42.1% | — | — | — | — |
| Depression | 22.6% | −0.27 | 0.12 | 4.79 | .029 * |
| Other | 35.3% | −0.14 | 0.11 | 1.76 | .185 |

Psychotropic Medication

| Prescribed Not Taking | 8.1% | | — | — | — | — |
|---|---|---|---|---|---|---|
| Prescribed Taking | 38.3% | | −0.04 | 0.18 | 0.04 | .841 |
| Not Prescribed | 52.5% | | 0.12 | 0.18 | 0.48 | .487 |
| Unknown/Not stated | 1.0% | | 0.02 | 0.48 | 0.002 | .969 |
| StartGad7 | 11.9 | | 0.14 | 0.05 | 7.87 | .005 ** |
| WaitingSAQ | 3.0 | days | 0.004 | 0.05 | 0.01 | .938 |
| WaitingAssignment | 0.8 | days | 0.001 | 0.06 | 0.0001 | .990 |
| WaitingContact | 1.1 | days | −0.14 | 0.08 | 2.84 | .092 |
| WaitingAssessment | 7.6 | days | −0.08 | 0.05 | 2.47 | .116 |
| WaitingTreatment | 8.9 | days | −0.002 | 0.05 | 0.001 | .972 |
| NumberSessions | 5.5 | sessions | 0.30 | 0.05 | 36.07 | <.001 *** |
| NumberDNA | 0.5 | sessions | −0.05 | 0.05 | 1.18 | .278 |

TABLE 10
Demographic details and clinical outcomes for patients finishing
a course of treatment between April 2015 and March 2016. The Internet-
enabled CBT cohort was severity matched to the IAPT cohort using a
multivariate rejection sampling. SD - standard deviation, n.a. - not
available. Diagnosis = “Depression” encompasses patients
diagnosed with depressive episode, dysthymia or recurrent depressive
disorder. Diagnosis = “Anxiety” encompasses patients
diagnosed with agoraphobia, generalised anxiety disorder, hypochondriacal
disorder, obsessive-compulsive disorder, panic disorder, post-traumatic
stress disorder, social phobia, specific phobia or anxiety disorder
unspecified. Diagnosis = “Other” encompasses all diagnoses
not otherwise listed (e.g.: chronic pain, bereavement, eating disorders).

| | | | | | | | Group |
|---|---|---|---|---|---|---|---|
| | Internet-enabled CBT | | | IAPT | | | difference |
| | (Severity matched) | | | (Areas where IECBT was | | | Odds ratio/ |
| | (N = 2,211) | | | offered) (N = 19,325) | | | Cohen's d |
| | Mean/Prevalence | SD | 95% CI | Mean/Prevalence | SD | 95% CI | (95% CI) |
| Gender | 73% Female | — | 71-75% | 67% Female | — | 67-68% | 1.30 |
| | | | | | | | (1.18-1.44) |
| Long Term | 21% | — | 20-23% | 23% | — | 22-23% | 0.92 |
| Physical | | | | | | | (0.83-1.03) |
| Conditions | | | | | | | |
| Diagnosis | | | | | | | |
| Depression | 27% | — | 25-29% | 36% | — | 35-36% | 0.68 |
| | | | | | | | (0.62-0.75) |
| Anxiety | 41% | — | 39-43% | 38% | — | 37-39% | 1.13 |
| | | | | | | | (1.03-1.24) |
| Other | 32% | — | 30-34% | 27% | — | 26-27% | 1.30 |
| | | | | | | | (1.18-1.43) |
| PHQ-9 at | 13.8 | 4.5 | 13.6-14.0 | 14.3 | 5.2 | 14.2-14.4 | −0.10 |
| assessment | | | | | | | (−0.14-−0.05) |
| GAD-7 at | 13.0 | 3.8 | 12.8-13.1 | 13.2 | 4.2 | 13.1-13.3 | −0.05 |
| assessment | | | | | | | (−0.09-0.004) |
| PHQ-9 | 8.6 | 6.1 | 8.3-8.9 | 8.5 | 6.2 | 8.4-8.6 | 0.02 |
| post-treatment | | | | | | | (−0.03-0.06) |
| GAD-7 | 8.0 | 5.4 | 7.8-8.2 | 7.8 | 5.4 | 7.7-7.9 | 0.04 |
| post-treatment | | | | | | | (−0.01-0.08) |
| Treatment | 5.5 sessions | 2.8 | 5.4-5.6 | 6.4 sessions¹ | n.a. | n.a. | n.a. |
| length | | | | | | | |
| Improvement | 65.8% | — | 63.8-67.8% | 66.2% | — | 65.5-66.8% | 0.98 |
| rate | | | | | | | (0.90-1.08) |
| Recovery rate | 48.8% | — | 46.7-51.0% | 49.5% | — | 48.8-50.3% | 0.97 |
| | | | | | | | (0.89-1.06) |

¹= average treatment duration for all conditions nationwide, regional data not available.

TABLE 11
Ranking of 21 anonymized, arbitrarily selected therapists based on effectiveness. Effectiveness
was measured by a comparison of observed overall recovery rate of all patients treated by
that therapist, with the predicted overall recovery rate for those patients calculated using
patient variables collected before treatment commenced. A positive value for observed ORR
minus expected ORR indicates the therapist performed better than expectation, and negative
value in that column indicates the therapist performed worse than expectation.

| | Anonymized | | | | | | Observed |
|---|---|---|---|---|---|---|---|
| | Therapist | Smoothed | Number of | Engagement | Observed | Expected | ORR minus |
| Rank | ID | p-value | cases seen | Rate | ORR | ORR | Expected ORR |
| 1 | 1E075551 | 0.02 | 13 | 1 | 0.846 | 0.467 | 0.379 |
| 2 | 95CB5326 | 0.025 | 23 | 0.826 | 0.609 | 0.385 | 0.224 |
| 3 | 635B1754 | 0.114 | 19 | 0.579 | 0.526 | 0.371 | 0.155 |
| 4 | F177861E | 0.252 | 13 | 0.692 | 0.462 | 0.338 | 0.123 |
| 5 | 179F1196 | 0.131 | 38 | 0.842 | 0.526 | 0.426 | 0.100 |
| 6 | 2F656FE3 | 0.055 | 87 | 0.644 | 0.517 | 0.43 | 0.087 |
| 7 | 01842DF4 | 0.438 | 10 | 0.6 | 0.3 | 0.231 | 0.069 |
| 8 | 73E1A916 | 0.094 | 127 | 0.709 | 0.441 | 0.384 | 0.057 |
| 9 | 9B75621E | 0.234 | 84 | 0.655 | 0.393 | 0.351 | 0.042 |
| 10 | 0F5B3B6F | 0.486 | 17 | 0.824 | 0.412 | 0.379 | 0.032 |
| 11 | 4BEC3178 | 0.42 | 53 | 0.83 | 0.415 | 0.393 | 0.022 |
| 12 | B4610B7C | 0.614 | 7 | 0.857 | 0.429 | 0.417 | 0.011 |
| 13 | 3117530F | 0.568 | 37 | 0.838 | 0.405 | 0.405 | 0 |
| 14 | C0EEB6F5 | 0.632 | 58 | 0.621 | 0.397 | 0.409 | −0.012 |
| 15 | 89E69734 | 0.662 | 18 | 0.5 | 0.333 | 0.352 | −0.019 |
| 16 | C6AC877A | 0.834 | 128 | 0.648 | 0.352 | 0.387 | −0.036 |
| 17 | 2218B6E1 | 0.77 | 23 | 0.826 | 0.261 | 0.306 | −0.045 |
| 18 | 06B7BC39 | 0.838 | 33 | 0.667 | 0.303 | 0.368 | −0.065 |
| 19 | AF9A035B | 0.887 | 42 | 0.643 | 0.333 | 0.411 | −0.077 |
| 20 | 1EEA1BC1 | 0.846 | 14 | 0.786 | 0.286 | 0.38 | −0.094 |
| 21 | C3DFE207 | 0.977 | 55 | 0.655 | 0.236 | 0.354 | −0.118 |

1. A method of determining the effectiveness of a therapist, comprising:
obtaining data relating to one or more patient variables and/or one or more service variables for one or more patient suffering from a mental health disorder and allocated to the therapist;
attributing a score to the data for each of the patient variables and/or the service variables, wherein the scores are based on a correlation between historic cohort treatment outcomes and historic cohort data comprising cohort patient and/or service variables;
combining the scores to calculate an aggregate score;
comparing the aggregate score with a scale to produce a prediction of psychological therapy outcome for the one or more patient;
obtaining an observation of psychological therapy outcome for the one or more patient after treatment by the therapist has been provided; and
comparing the observation of psychological therapy outcome and the prediction of psychological therapy outcome for the one or more patient to make a determination of the effectiveness of the therapist.

2. The method of claim 1, further comprising taking an action based on the determination of the effectiveness of the therapist.

3. The method of claim 2, wherein the action comprises an action selected from the group consisting of (1) providing additional training materials to the therapist, (2) initiating additional supervision of the therapist, (3) initiating further training of the therapist, and (4) reallocating patients from the therapist to one or more other therapist.

4. A method of treating a patient comprising:
obtaining data relating to one or more patient variables and/or one or more service variables for a patient suffering from a mental health disorder;
attributing a score to the data for each of the patient variables and/or the service variables, wherein the scores are based on a correlation between historic cohort treatment outcomes and historic cohort data comprising cohort patient and/or service variables;
combining the scores to calculate an aggregate score;
comparing the aggregate score with a scale to produce a prediction of psychological therapy outcome for the patient; and
treating the patient according to a treatment protocol determined based on a comparison of the prediction of psychological therapy outcome for the patient and one or more criteria derived from the correlation between the historic cohort treatment outcomes and the historic cohort data.

5. The method of claim 4, wherein the treatment protocol comprises one or more treatments selected from the group consisting of (1) a specified frequency of one-to-one or face-to-face meetings, (2) a specified frequency of asynchronous messaging, (3) the provision of self-help materials, (4) an indication of a potential need for psychotropic medication(s), and (5) the allocation of a particular therapist.

6. The method of claim 4 further comprising:
assessing a general anxiety disorder 7-item (GAD-7) score and/or a patient health questionnaire (PHQ-9) score before implementing the psychological therapy, wherein the GAD-7 score and/or the PHQ-9 score is included as one or more of the patient variables.

7. The method of claim 4, wherein the psychological therapy comprises internet-enabled cognitive behavioural therapy.

8. The method of claim 4, wherein the mental health disorder comprises a disorder selected from the group consisting of (1) depression, (2) mixed anxiety and depression, (3) generalized anxiety disorder, (4) social phobias, (5) panic disorder, (6) obsessive-compulsive disorder, (7) post-traumatic stress disorder, (8) agoraphobia, (9) specific phobias, and (10) another anxiety disorder.

9. The method of claim 4, wherein the one or more patient variables comprises a variable selected from the group consisting of (1) patient gender, (2) patient age, (3) whether or not the patient suffers from a long-term physical condition, (4) whether or not the patient is taking psychotropic medication at the start of treatment, (5) the initial symptom severity, (6) the mental health disorder the patient suffers from, (7) whether or not a patient is currently pregnant, or has been pregnant or given birth in the previous 12 months, and (8) patient employment status.

10. The method of claim 4, wherein the one or more service variables comprises a variable selected from the group consisting of (1) waiting times between various stages in the patient journey, (2) treatment duration, (3) the number of scheduled appointments the patient fails to attend, (4) the therapist the patient is allocated to, and (5) the treatment protocol the patient receives.

11. The method of claim 4, wherein the prediction of psychological therapy outcome for the patient is a measure of improvement and/or a measure of recovery.

12. The method of claim 4, further comprising:
performing the method at two times during the psychological therapy to attain a first prediction of psychological therapy outcome for the patient and a second prediction of psychological therapy outcome for the patient;
comparing the first and second prediction of psychological therapy outcome for the patient; and
using the comparison of first and second prediction of psychological therapy outcome to calculate a measure of quality of the psychological therapy.

13. The method of claim 12 further comprising:
calculating a reimbursement value to the psychological therapy in a fee-for-value payment system based on the measure of quality of the psychological therapy.

14. The method of claim 4, further comprising:
obtaining second data relating to the one or more patient variables and/or the one or more service variables for the patient at a time after beginning the treatment protocol;
attributing a second score to the second data for each of the patient variables and/or the service variables;
combining the second scores to calculate a second aggregate score;
using the second aggregate score to make a second prediction of psychological therapy outcome for the patient; and
treating the patient according to a second treatment protocol determined based on the second prediction of psychological therapy outcome for the patient.

15. The method of claim 4, further comprising:
determining whether the patient suffers from a long-term physical condition; and
treating the patient according to a treatment protocol determined based on both the prediction of psychological therapy outcome for the patient and the determination of whether the patient suffers from a long-term physical condition.

16. A non-transitory, tangible, computer-readable storage medium: containing a program of instructions that cause a computer system running the program of instructions to:
receive data relating to one or more patient variables and/or one or more service variables for one or more patient suffering from a mental health disorder and allocated to the therapist;
attribute a score to the data for each of the patient variables and/or the service variables, wherein the scores are based on a correlation between historic cohort treatment outcomes and historic cohort data comprising cohort patient and/or service variables;
combine the scores to calculate an aggregate score;
compare the aggregate score with a scale to produce a prediction of psychological therapy outcome for the one or more patient;
receive further data relating to an observation of psychological therapy outcome for the one or more patient after treatment by the therapist has been provided; and
compare the observation of psychological therapy outcome and the prediction of psychological therapy outcome for the one or more patient to make a determination of the effectiveness of the therapist.

17. The medium of claim 16, wherein the program of instructions further cause the computer system running the program of instructions to:
take an action based on the determination of the effectiveness of the therapist.

18. The method of claim 17, wherein the action comprises an action selected from the group consisting of: (1) providing additional training materials to the therapist, (2) initiating additional supervision of the therapist, (3) initiating further training of the therapist, and (4) reallocating patients from the therapist to one or more other therapist.

19. The medium of claim 17, wherein the received data is from more than one hardware source, and optionally wherein the hardware source comprises a mobile device.
